(19) United States
(12) Patent Application Publication
(10) Pub. No.: US 2024/0061002 A1
(43) Pub. Date: Feb. 22, 2024

(54) PATHOGEN TESTING SYSTEMS AND METHODS OF USE THEREOF

(71) Applicants: Salus Discovery, LLC, Madison, WI (US); Flambeau Diagnostics, LLC, Monona, WI (US); Wisconsin Alumni Research Foundation, Madison, WI (US)

(72) Inventors: Peter James Dailey, Clayton, CA (US); Richard McGregor Thayer, Meridian, ID (US); Laura Terese Mazzola, San Francisco, CA (US); Paul Scott Eastman, Danville, CA (US); Michael S. Urdea, Alamo, CA (US); Jesse Charles Darley, Madison, WI (US); Ryan Lee Anton, Madison, WI (US); Katherine Gail Mason, Plain City, OH (US); Sara Willoughby Berg-Love, Loudonville, NY (US); Justin Daniel Chan, Middleton, WI (US); Russell Prince, Middleton, WI (US); Douglas Paul Barnes, San Francisco, CA (US); Evan Alex Bunke, Madison, WI (US); Jacob Emory Schepp, Marshall, WI (US); Douglas E. Seals, Fitchburg, WI (US); Alexandra A. Surasky-Ysasi, Madison, WI (US); Angie Purvis, Evergreen, CO (US); Christina Newman, Madison, WI (US); Dawn Dudley, Madison, WI (US); David O'Connor, Madison, WI (US); Thomas Friedrich, Madison, WI (US); Steven Carbone, Woburn, MA (US); Madalyn Gill, Madison, WI (US); Brianna Mullins, Deforest, WI (US); Jay Warrick, Madison, WI (US); Patrick McMinn, Madison, WI (US); Dave Beebe, Monona, WI (US); Ryan Shogren, Madison, WI (US); Mitch Geiger, Madison, WI (US); Franklin Zhong, Madison, WI (US); Duane S. Juang, Madison, WI (US); Terry D. Juang, Madison, WI (US)

(21) Appl. No.: 18/270,438

(22) PCT Filed: Jan. 5, 2022
(86) PCT No.: PCT/US2022/011304

§ 371 Date: Jun. 29, 2023

Related U.S. Application Data
(60) Provisional application No. 63/134,463, filed on Jan. 6, 2021.

(51) Int. Cl. G01N 35/00; G01N 35/10; C12Q 1/70; B03C 1/01; B03C 1/28

(52) U.S. Cl. CPC G01N 35/0098; G01N 35/0099; G01N 35/1011; C12Q 1/701; B03C 1/01; B03C 1/288; B03C 2201/26; B03C 2201/18

(57) ABSTRACT

Provided herein are mobile systems for sample processing. In some aspects, provided herein are mobile systems for sample processing and methods of use thereof for detection of pathogens in biological samples.

### STATEMENT REGARDING RELATED APPLICATIONS

This application claims priority to U.S. Provisional Patent Application No. 63/134,463, filed Jan. 6, 2021, the entire contents of which are incorporated herein by reference for all purposes.

### STATEMENT OF GOVERNMENT SUPPORT

This invention was made with government support under AI132132, OD011106, and R43 OD023021-01A1, awarded by the National Institutes of Health. The government has certain rights in the invention.

### FIELD

Provided herein are systems and methods of use thereof for detection of pathogens in biological samples.

### BACKGROUND

Diagnostic testing and treatment for infection typically requires that the potentially infected individual go to a clinic or testing facility in order to have a biological sample collected for testing, thus potentially exposing others to the same infection. Similarly, rapid turnaround of test results and treatment are key to quarantining infected individuals in a timely manner before they spread virus to others. Removing logistical constraints for testing and treatment is a key way to reduce that turnaround time. In the wake of the global COVID-19 pandemic, improved systems for providing rapid and accurate diagnostic testing and treatment while minimizing risk for transmission of infection are of utmost importance.

### BRIEF SUMMARY

The inventions described and claimed herein have many attributes and embodiments including, but not limited to, those set forth or described or referenced in this Brief Summary. It is not intended to be all-inclusive, and the inventions described and claimed herein are not limited to or by the features or embodiments identified in this introduction, which is included for purposes of illustration only and not restriction.

In some aspects, provided herein are mobile, movable and semi-permanent laboratories for processing biological samples. In some embodiments provided herein are mobile laboratories in vehicles. In some embodiments provided herein is a mobile laboratory comprising a computer system comprising one or more processors. In some embodiments, the computer system is a computer readable storage medium having computer instructions stored therein is provided, where once the computer instructions are executed, the disclosed methods for processing biological samples are performed.

In some embodiments, a mobile sample processing and assay system including a memory and a processor is provided, where the memory has computer instructions stored therein, and once the processor executes the computer instructions, a method disclosed herein for processing a sample, isolating a target, and assaying the sample for the target system is performed.

The processors may be configured to catalog a plurality of biological samples collected from distinct subjects, operate a robotic sample processing system, operate a robotic sample analysis system, associated data received from the sample analysis system with individual members of the plurality of biological samples, and store, depict, output and locally or remotely communicate said data. In some embodiments, the mobile laboratory further comprises a robotic sample processing system. The robotic sample processing system may be configured to mix each biological sample containing or suspected of containing a target with magnetic target capture particles (e.g., paramagnetic particles (PMPs) or ferromagnetic particles) to generate a composition comprising one or more target-PMP complexes, for example, and separate or isolate the target-PMP complexes from each composition. The mobile laboratory may further comprise a robotic sample analysis system. In some embodiments, the robotic sample analysis system is configured to receive the target-PMP complexes and carry out an assay, for example, a biological assay to detect said target, if present.

In some embodiments, biological samples processed using the mobile laboratories described herein comprise nasopharyngeal samples, oropharyngeal samples, oral swab samples, oral sponge samples, nasal swab samples, mid-turbinate samples, or saliva samples. In some embodiments, each biological sample is stored in a sample storage container (e.g., a tube). In some embodiments, each sample storage container comprises a storage buffer. In some embodiments, each sample storage container is heated to at least 40° C. after placement of the biological sample within the tube.

In some embodiments, the biological sample additionally comprises a reducing agent. In some embodiments, the reducing agent is dithiothreitol (DTT), tris(2-carboxyethyl)phosphine (TCEP), or 2-mercaptoethanol.

In some embodiments, the biological sample additionally comprises a protease. For example, the biological sample may additionally comprise Proteinase K.

In some embodiments, magnetic target capture particles (e.g., paramagnetic particles (PMPs)) are contained in a liquid composition. In some embodiments, the liquid composition containing the PMPs, for example, additionally comprises DTT. In some embodiments, the liquid composition containing the PMPs additionally comprises a detergent. In other embodiments, the PMPs are contained in a lyophilized formulation.

In some embodiments, the robotic sample processing system comprises a multichannel pipette and an apparatus for operating the multichannel pipette. In some embodiments, the apparatus for operating the multichannel pipette is configured to induce movement of the multichannel pipette. In some embodiments, the apparatus for operating the multichannel pipette is configured to aspirate and/or inject liquid when pipette tips are attached to the multichannel pipette. The apparatus for operating the multichannel pipette may be controlled by the computer system.

In some embodiments, the robotic sample processing system is configured to mix each biological sample with magnetic particles, such as paramagnetic particles (PMPs) or ferromagnetic particles, to generate a composition comprising, for example, one or more target-PMP complexes. In some embodiments, mixing each biological sample with PMPs, for example, is performed in a multi-well plate using a plurality of pipette tips attached to the multichannel pipette.

In some embodiments, referring to PMPs and pipette tips, the robotic sample processing system is configured to separate or isolate the one or more target-PMP complexes from each composition. Many suitable methods for isolating target-PMP complexes are described herein. In some embodiments, isolating target-PMP complexes from each composition (e.g. each composition comprising the one or more target-PMP complexes) comprises aspirating each composition into a distinct pipette tip and positioning a multichannel pipette proximal to a magnet positioned below a sample collection device to provide a magnetic force, such that the magnet draws the target-PMP complexes out of the pipette tips and into the sample collection device. The magnetic force may draw less than all of the target-PMP complexes out of the pipette tips and into the sample collection device for qualitative or semi-quantitative target determinations. The magnetic force may draw most or all target-PMP complexes out of the pipette tips and into the sample collection device for quantitative target determinations.

In some embodiments, separating or isolating the target-PMP complexes from each composition comprises aspirating each biological sample into a distinct pipette tip containing lyophilized PMPs, for example, to generate a composition comprising one or more target-PMP complexes within each pipette tip. Following generation of the target-PMP complexes, the multichannel pipette may be positioned proximal to a magnet positioned below a sample collection device to provide a magnetic force, such that the magnet draws target-PMP complexes out of the pipette tips and into the sample collection device.

In some embodiments, separating or isolating the target-PMP complexes involves generating a liquid/air interface, liquid/oil interface, or hybrid interface proximal to a bottom opening of the pipette tip. A magnetic force may then be used to draw the target-PMP complexes through the interface, such as through the interface and into a sample collection device. In some embodiments, isolating the target-PMP complexes from each composition comprises aspirating each composition into a distinct pipette tip, generating a liquid/air interface, a liquid/oil interface, or a hybrid interface proximal to a bottom opening of the pipette tip, and positioning the multichannel pipette proximal to a magnet positioned below a sample collection device, such that the magnet draws the target-PMP complexes through the liquid/air interface, liquid/oil interface, or hybrid interface and into the sample collection device.

In some embodiments, the methods described herein involve a preconcentration step in which pipette tips holding the composition comprising the plurality of biological samples are placed in proximity to a first magnetic force (e.g., a first magnet) to generate a concentration of target-PMP complexes proximal to the liquid/air interface, liquid/oil interface, or hybrid interface of the pipette tip. For example, in some embodiments isolating target-PMP complexes from each composition comprises aspirating each composition into a distinct pipette tip, generating a liquid/air interface, a liquid/oil interface, or a hybrid interface proximal to a bottom opening of the pipette tip, positioning the multichannel pipette proximal to a first magnet to generate a concentration of target-PMP complexes proximal to the liquid/air interface, liquid/oil interface, or hybrid interface, and positioning the multichannel pipette proximal to a second magnetic force (e.g., a second magnet) to draw target-PMP complexes through the liquid/air interface, liquid/oil interface, or hybrid interface and into a sample collection device.

In some embodiments, isolating target-PMP complexes from each composition comprises aspirating each biological sample into a distinct pipette tip containing lyophilized PMPs to generate a composition comprising one or more-target PMP complexes within each pipette tip, generating a liquid/air interface, a liquid/oil interface, or a hybrid interface proximal to a bottom opening of the pipette tip, and positioning the multichannel pipette proximal to a magnet positioned below a sample collection device, such that the magnet draws the target-PMP complexes through the liquid/air interface, liquid/oil interface, or hybrid interface and into the sample collection device.

In some embodiments, isolating the target-PMP complexes from each composition comprises aspirating each biological sample into a distinct pipette tip containing lyophilized PMPs to generate a composition comprising one or more-target PMP complexes within each pipette tip, generating a liquid/air interface, a liquid/oil interface, or a hybrid interface proximal to a bottom opening of the pipette tip, positioning the multichannel pipette proximal to a first magnet to generate a concentration of target-PMP complexes proximal to the liquid/air interface, liquid/oil interface, or hybrid interface, and positioning the multichannel pipette proximal to a second magnet to draw the target-PMP complexes through the liquid/air interface, liquid/oil interface, or hybrid interface and into a sample collection device.

In some embodiments, each composition or biological sample is aspirated into a distinct pipette tip through the bottom opening of the pipette tip. In such embodiments, generating the liquid/air interface, liquid/oil interface, or hybrid interface comprises further aspirating the composition within the pipette tip while the bottom opening of the pipette tip is exposed to air. In other embodiments, each composition or biological sample is aspirated into a distinct pipette tip through a side opening of the pipette tip while the bottom opening of the pipette tip is in conformal contact with a surface such that liquid is unable to enter the pipette tip through the bottom opening, thereby generating the liquid/air interface, liquid/oil interface, or hybrid interface.

In some embodiments, the sample collection device comprises a multi-well plate, such that the target-PMP complexes from each composition are drawn into a distinct well on the multi-well plate. In some embodiments, the distinct wells on the multi-well plate comprise a wash buffer. In some embodiments, processing the plurality of biological samples further comprises aspirating the wash buffer from the wells and allowing the target-PMP complexes contained therein to dry.

In some embodiments, the systems described herein further comprise a moveable surface, wherein the biological samples, one or more magnets, and/or the sample collection device are housed on the moveable surface. In some embodiments, the moveable surface is configured to change orientation and/or move in the x-y plane and or in the vertical z-direction.

In some embodiments, wherein the assay comprises reagents for loop-mediated isothermal amplification (LAMP)-based detection of the target. In some embodiments, the target comprises viral nucleic acid. In some embodiments, the target comprises viral nucleic acid from an upper respiratory infection selected from SARS-CoV2, coronavirus, rhinovirus, influenza, respiratory syncytial virus, adenovirus, parainfluenza, human immunodeficiency virus, human papillomavirus, rotavirus, hepatitis C virus, zika virus, Ebola virus, tuberculosis, Borrelia burgdorferi, Staphylococcus, Aspergillus, Streptococcus pyogenes. In some embodiments, the target comprises SARS-CoV2 nucleic acid.

In some embodiments, isolated or separated target-PMP complexes are contacted with the reagents for LAMP-based detection of the target, and a signal resulting from contact is measured. For example, the signal may be a colorimetric signal or a fluorescent signal. Data regarding detection of the target may comprise data regarding the signal. In some embodiments, data regarding detection of the target is correlated with a unique identifier for each biological sample. In some embodiments, the mobile laboratory is housed within a vehicle. In some embodiments, the mobile laboratory is housed within a building for a predetermined period, for example, a period of less than 6 months prior to being moved to a new location.

### BRIEF DESCRIPTION OF THE DRAWINGS

FIG. 1A shows time to threshold values for contrived saliva samples containing 10⁴ viral copies/mL.

FIG. 2A shows time to threshold values for contrived saliva samples obtained from 7 different subjects. DTT was added to the samples before or after heating the samples, and time to threshold values were compared, showing that the addition of a reducing agent before the heat lysis step decreased the time to threshold values for each sample. FIG. 2B also shows improved time to threshold values for virion controls subjected to the same saliva treatment, air purification, and LAMP analysis steps as above.

FIG. 3A shows a heatmap of saliva or NP samples processed with different sample buffer reagents. FIG. 3B shows the results for the same combinations, quantified as time to threshold values.

FIG. 4A shows time to threshold values for NP samples in various buffers (UTM, PBS, or saline). Time to threshold values for NP virion controls in saline are shown in FIG. 4B.

FIG. 5 shows a schematic of an exemplary embodiment described herein wherein a hybrid interface is created. Biological samples containing a virus are mixed with a liquid composition comprising PMPs (e.g. mag beads) and a detergent (e.g. lysis buffer). A pipette tip is dipped in oil (step 1), and the mixture of the biological sample and liquid composition is aspirated into the tip (step 2). An air gap is created (step 3), and a magnet is used to concentrate the target:PMP complexes near the air gap (step 4). The target:PMP complexes are drawn through the air pocket into a multi-well plate containing a wash buffer by a second magnet (step 5). The wash buffer is aspirated from the wells (step 6) and LAMP reagents are added to the wells (step 7). The target:PMP complexes and LAMP reagents are mixed and an oil overlay is placed on top of each well (step 8). The multi-well plate is placed in a plate reader for incubation and detection (e.g. colorimetric detection, step 9).

FIG. 6 shows an exemplary embodiment using a tip designed to have a side opening, such that the liquid/air interface can be generated during aspiration of the biological sample.

FIG. 7 shows an exemplary layout of a system that may be used to perform the methods for isolating a target described herein. The system is shown prior to placement of the proper components within the system.

FIG. 8 shows an exemplary layout of a system that may be used to perform the methods for isolating a target described herein (FIG. 8A). The system is shown after placement of the proper components within one example of the system, where 1 is a Waste Chute, 2 is a Mixing Plate, 3 is a Reagent Source Plate, 4 is a Tip Box, 5 is a Sample Rack, 6 and 7 are Tip Boxes, 8 is a Reaction Plate and 9 is a Tip Box (FIG. 8B).

FIG. 9 shows exemplary means to achieve sufficient power to the mobile laboratory described herein. These means may be particularly well suited for a mobile laboratory contained within a vehicle, such as a van. In some embodiments, the vehicle may be uplifted with an additional alternator and an upgraded battery Such a strategy may provide sufficient power to the mobile laboratory, while also removing the need for a gas-powered generator or shore power. However, such a mobile laboratory may also be compatible with shore power. In some embodiments, the mobile laboratory may instead be equipped with batteries, including pre-charged batteries. If uplifting of the vehicle (e.g. van) is not possible, an external generator may be used, such as in rural settings, while shore power may be a viable solution in more urban settings, for example.

FIG. 10 shows an exemplary layout of the ancillary equipment which, combined with the components shown in FIG. 11, comprise a mobile laboratory that can be housed within a vehicle, such as a van, for example. The Figures demonstrate the layout of the interior of the van which houses the mobile laboratory and all of the necessary equipment for high-throughput testing, such as high-throughput SARS-Cov-2 nucleic acid testing, for example. This layout was developed in consideration of industrial, electrical, and software requirements to optimize both workflow and space efficiency, as well as safety. In some embodiments, ovens may remain proximal to the door of the vehicle to minimize penetration of raw samples into the workspace, and maximize separation between the clean assay work area and a potentially contaminated workspace. Organizers of disposable items (blue area with white and gray grid patterns) may be located near the automated sample processing unit for isolating target-PMP complexes, for example (to the left). A waste chute may be used to limit potential contamination from the waste bin (shown in red). A standing workstation may be used to maximize space within the van while providing mobility for technician activities.

FIGS. 11A and 11B show exemplary renderings of a mobile laboratory, also sometimes referred to as the Lab Automation Cell, or LAC. Such a mobile laboratory may be housed within a vehicle or within a building or other semi-permanent structure. A modular frame structure was selected to house automation components (e.g. the components of the mobile laboratory). In this embodiment, 80/20 frame components were used that allow support structures to be placed anywhere within the confines of the frame. This provided infinite flexibility for arranging the automation components. Accordingly, as needs for different assays may change, the infrastructure is able to be reconfigured easily to adapt. In the case of using the mobile laboratory to perform a rapid SARS-CoV-2 test, for example, one liquid handler, one plate sealer, two dual function plate readers (absorbance/fluorescence), 2 waste bins, and a 6-axis robotic arm may be used as shown in the Figure. The layout depicted, with the robotic arm hanging from the ceiling, provided the necessary space efficiency to the frame to fit through standard building doorways as well as within the height restrictions of a van while the base of the frame can be configured to mount semi-permanently to a surface or be put on wheels for manual transportation. The overall dimensions of the LAC in FIG. 11B (height×length×depth) are 1.75 meters×1.55 meters×0.88 meters (68.9 inches×60.6 inches×34.6 inches).

FIG. 12A-D show several exemplary user interfaces that may be used to track sample processing by the mobile laboratory disclosed herein. The user interface may be a component within the system, such as present within the LAC, the vehicle or present within the building in which the mobile laboratory is placed. FIG. 12A shows key functions achieved by a user interface. Functions comprise: to guide the user through steps using spatial layout out checklist organization, login all events to an event log, provide the ability to see the current action and preview or be warned of upcoming actions, notify at the moment an action becomes available with multiple urgency levels, and communicate whether the machine cycle is in progress or completed. FIGS. 12 B, 12C, and 12D show an exemplary user interface with various messages displayed therein.

FIG. 13 shows an exemplary design for a robotic component that may be used to move components of the mobile laboratory, such as multi-well plates. This particular robotic component is referred to herein as “grippers”. Typically, as the fingers of the grippers travel inward, the mounting points interfere with each other limiting their travel. This is important when considering that the handling of assay plates must sometimes be done by grabbing them across their long dimension (127.76 mm), and sometimes from their short direction (85.48 mm). If the travel of the grippers is limited, it is not possible to grab from both directions with the same gripper design. Thus, alteration of gripper details were made to allow the motors to bring the details closer together, significantly extending the travel. The extended travel allowed for gripping plates from both the long and short dimension, thereby providing total flexibility using a small, space efficient gripper with limited travel.

### DEFINITIONS

To facilitate an understanding of the present disclosure, a number of terms and phrases are defined below:

As used herein, “computer system,” “computing device,” “computer,” and analogous expressions refer to one or more devices including at least one tangible computing element. Examples of a tangible computing element include but are not limited to a microprocessor, application specific integrated circuit, programmable gate array, and the like. Examples of a computing device include but are not limited to a mobile computing device such as a smart phone or tablet computer, a wearable computing device (e.g., smart glasses), a laptop computer, a desktop computer, or any other form of computing device. A computing device preferably includes or accesses storage for instructions used to perform steps such as those described herein, access samples or data on which those steps may be performed, and access reagents and materials.

As used herein, “computer-implemented” and analogous expressions refer to technology implemented using, by, with or on one or more computing systems or computer devices.

As used herein, “cause to be displayed,” “causing to be displayed” and analogous expressions refer to taking one or more actions that result in displaying. A computing device, under control of program code, may cause to be displayed an image, picture and/or text for example, and other information to any user of the computing device. For example, a computing device may cause images, pictures, graphs and/or texts—including sample information, patient information, system information, data and test results, etc. —to be displayed directly to a user. For another example, a local or remote server computer under control of program code may cause information to be displayed by making the information available for access by a local computer or mobile device, for example, over a network, such as the Internet, which information the local computer or mobile device may then display to a user of the computer or the mobile device.

As used herein, the terms “detect”, “detecting”, or “detection” may describe either the general act of discovering or discerning or the specific observation of a detectably labeled composition. The term “detecting” when used in reference to a target refers to detecting either the presence or the absence of the target in the sample. In some embodiments, “detecting” a target in a sample refers to determining that the target is present in the sample. In some embodiments, “detecting” a target in a sample refers to determining that the target is not present in the sample or is not present in sufficient quantities to be detected in the sample.

As used herein, the term “biological sample” is used in the broadest sense and is inclusive of many sample types that may be obtained from a subject. Biological samples may be obtained from animals (including humans) and encompass fluids (e.g. urine, blood, blood products, sputum, saliva, etc.), solids, tissues, and gases. Biological samples include saliva, blood products, such as plasma, serum and the like. In some embodiments, the biological sample is a nasopharyngeal sample, an oropharyngeal sample, oral swab or sponge sample, a nasal swab sample, a mid-turbinate sample, or a saliva sample. In some embodiments, the biological sample is a saliva sample. The term “saliva sample” as used herein refers to a sample of saliva collected from a subject. In some embodiments, the biological sample is a nasopharyngeal (NP) sample. A “nasopharyngeal sample” refers to a specimen collected using a swab inserted into the nasopharyngeal cavity of a subject. The biological sample may be subjected to various pre-treatment steps prior to performing a method as described herein. For example, the biological sample may be frozen, heated, mixed with various denaturants (e.g. guanidium thiocyanate), mixed with viscosity reducing reagents (e.g. DTT), mixed with inhibitors of target degradation (e.g. protease inhibitors, RNAse inhibitors, etc.), mixed with various buffers, or subjected to other suitable pre-treatment steps. In some embodiments, samples contain or are suspected of containing a microorganism (e.g. a pathogenic or disease-causing microorganism). Any of the denaturants, viscosity reducing reagents, inhibitors of target degradation, buffers, etc. may be added to the biological sample at a suitable point in time or may be present in a storage buffer (e.g. a storage buffer in a sample storage container) to which the biological sample is added at the time of collection.

The term “mobile” as used herein refers things that are not fixtures or specially built or installed permanently for use with a property, other than a vehicle. The term “vehicles” refers to any conveyance used for transporting passengers and/or things by land, water, or air. Vehicles include motor vehicles (motorcycles, cars, trucks, buses, ATVs, etc.), railed vehicles (trains, trams, etc.), watercraft (ships, boats, etc.), aircraft (airplanes, helicopters, aerostats, aerodynes, etc.), wagons, carts, 2- and 3-wheeled motorized and self-propelled devices useful for transporting a laboratory as described herein. In some embodiments, a mobile laboratory in a van or truck is provided.

The term “mobile laboratory” as used herein refers to a set of equipment that can be efficiently relocated from location to location and provides the functionality necessary to support delivery of testing (e.g., diagnostic testing) or medical services (e.g., vaccinations). In some embodiments, the mobile laboratory described herein comprises a computer system, a robotic sample processing system, and a robotic sample analysis system that are housed together in or on a moveable structure. For example, the mobile laboratory (e.g. the mobile laboratory comprising a computer system, a robotic sample processing system, and a robotic sample analysis system) may be housed on or within a structure with wheels or wings. For example, the mobile laboratory may be housed within a vehicle, such as a motor vehicle or airplane. As another example, the mobile laboratory may be housed on or within a cart, and the cart may be transported from one physical location to another. In such embodiments, the structure with wheels (e.g. vehicle, cart, etc.) can be moved, thus transporting the mobile laboratory from one location to another. In some embodiments, computer system support for the mobile laboratory is provided remotely, e.g., by wireless or other remote technology, while a robotic sample processing system, a robotic sample analysis system and other parts of the mobile laboratory are housed together on or within a moveable structure, e.g., a vehicle. Various diagnostic methods for use in a mobile laboratory are described herein.

The term “subject” as used herein refers to an entity from which a biological sample is obtained, directly or indirectly. The subject may be a mammal. In some embodiments, the subject is a human.

The term “target” as used herein is used in the broadest sense and refers to any desired material that may bind a magnetic particle (e.g., a paramagnetic particle or a ferromagnetic particle) and be pulled, wholly or partially, from a biological sample by application of a magnetic force. In some embodiments, the target is a protein or peptide (e.g. antibody, receptor, enzyme, hormone or other messenger protein or peptide), whole cell, or a nucleic acid (e.g. DNA, RNA). In some embodiments, the target is a metabolite, a carbohydrate, a glycopeptide, or a lipid.

### DETAILED DESCRIPTION

It is to be understood that the inventions are not limited to the particular devices, methodology, protocols, constructs, and reagents described herein and as such may vary. It is also to be understood that the terminology used herein is for the purpose of describing particular embodiments only, and that the scope of the inventions is limited only by the appended claims. As used herein and in the appended claims, the singular forms “a,” “an,” and “the” include plural reference unless the context clearly indicates otherwise. Unless defined otherwise, all technical and scientific terms used herein have the same meaning as commonly understood to those of ordinary skill in the art to which the inventions belong. Although any method steps, devices, and materials equivalent to those described herein can be used in the practice of the inventions, examples of methods, devices and materials for use in the practice of the inventions are now described.

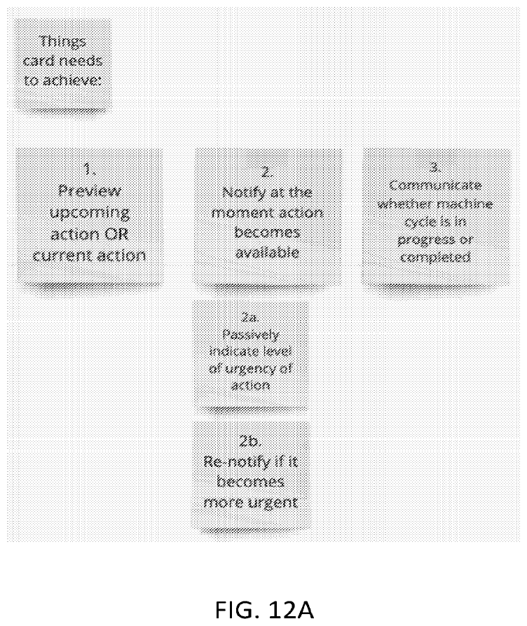

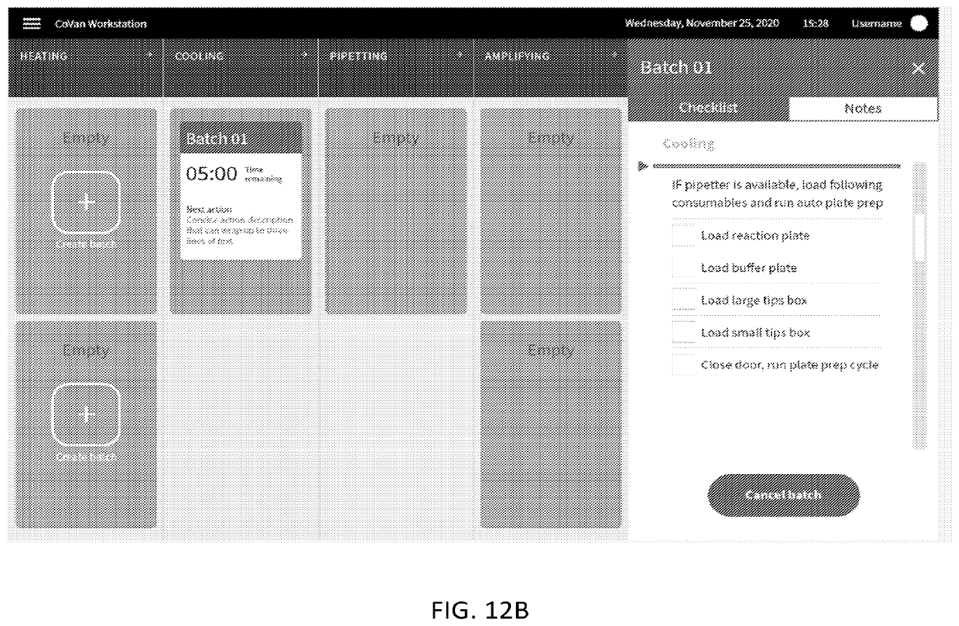

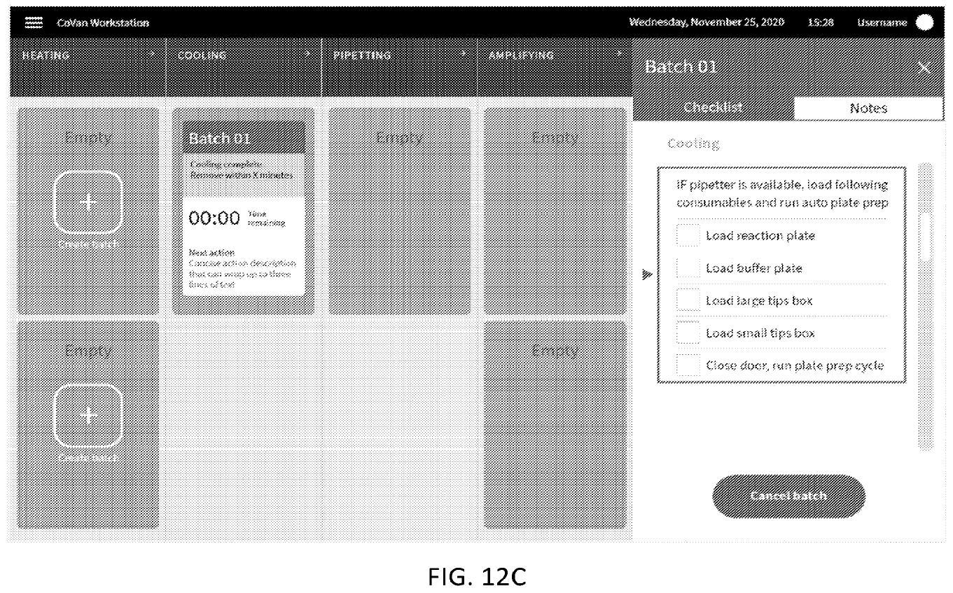

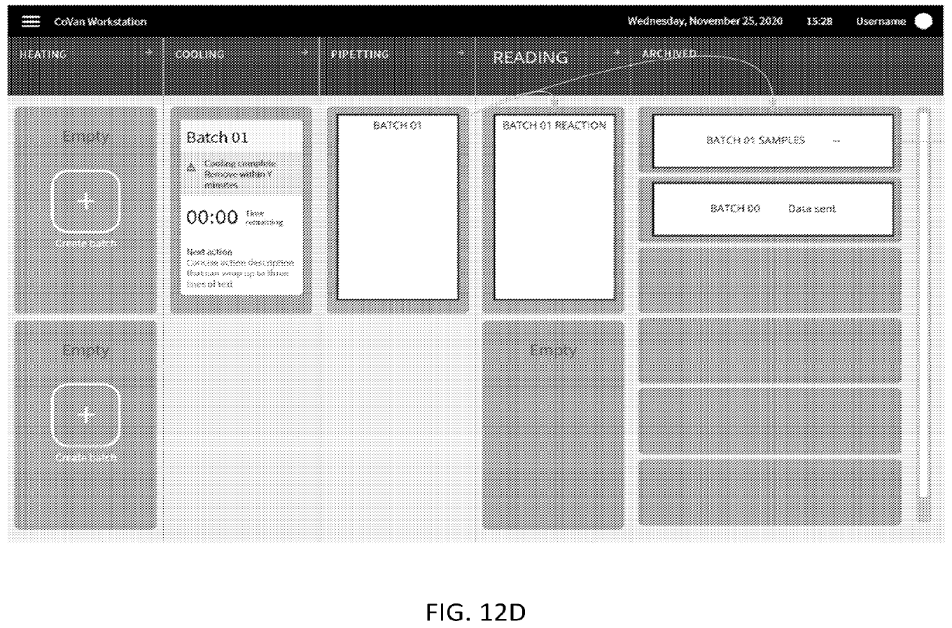

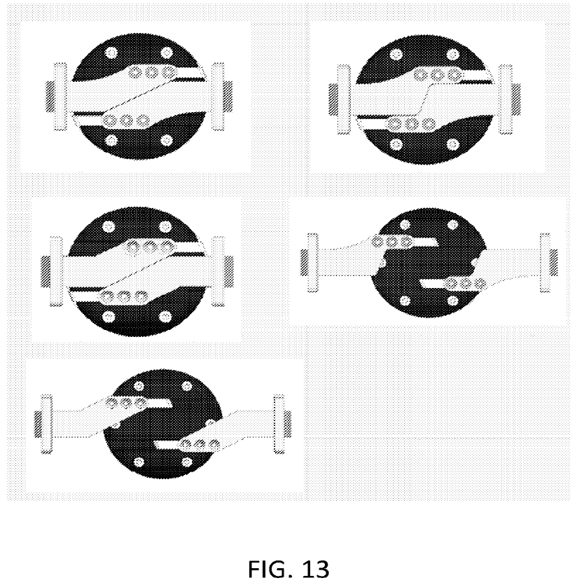

Provided herein are systems for sample processing. In some aspects, provided herein are systems for sample processing and methods of use thereof for detection of pathogens in biological samples. In some embodiments, the systems comprise a mobile laboratory containing a computer system, a robotic sample processing system, and a robotic sample analysis system. The computer system may comprise one or more processors configured to: i) catalog a plurality of biological samples collected from distinct subjects; ii) operate the robotic sample processing system; iii) operate the robotic sample analysis system; iv) associated data received from the sample analysis system with individual members of said plurality of biological samples; and v) locally and/or remotely communicate said data. In some embodiments, the robotic sample processing system is configured to (i) mix each biological sample with paramagnetic particles (PMPs) to generate a plurality of compositions, wherein each composition comprises one or more target-PMP complexes, and (ii) isolate the target-PMP complexes from each composition. In some embodiments, the robotic sample analysis system is configured to receive isolated target-PMP complexes and carry out an assay, for example, a biological assay to detect said target, if present. In some embodiments, the systems comprise a user interface to allow personnel to track and sample processing. For example, the system may comprise a user interface that, amongst other things, causes information to be displayed, including, for example, demonstrating current stages of sample processing (e.g. whether a batch of samples is being heated, cooled, mixed with PMPs, undergoing target-PMP complex isolation steps, etc.), signifying personnel interacting with the mobile laboratory about current steps, future steps, actions that need to be taken by the personnel, etc. Exemplary features of a user interface, and several examples of user interfaces are shown in FIGS. 12-A-D.

Embodiments of the present disclosure include various steps and operations, which are described herein. A variety of these steps and operations may be performed by hardware components or may be embodied in machine-executable instructions, which may be used to cause a general-purpose or special-purpose processor programmed with the instructions to perform the steps. Alternatively, the steps may be performed by a combination of hardware, software, and/or firmware.

In some embodiments, the sample processing system comprises one or more processors; and a computer readable storage medium having instructions stored thereon, which when executed by the one or more processors cause the sample processing system to carry out the sample preparation, sample processing and assay methods described herein. The methods and techniques described herein can be embodied as special-purpose hardware (e.g., circuitry), as programmable circuitry appropriately programmed with software and/or firmware, or as a combination of special-purpose and programmable circuitry. Hence, embodiments may include a machine-readable medium having stored thereon instructions that may be used to program a computer (or other electronic devices, including phones or tablets) to perform a process. The machine-readable medium may include, but is not limited to, floppy diskettes, optical discs, compact disc read-only memories (CD-ROMs), magneto-optical discs, read-only memories (ROMs), random-access memories (RAMs), erasable programmable read-only memories (EPROMs), electrically erasable programmable read-only memories (EEPROMs), magnetic or optical cards, flash memory, or other types of machine-readable media suitable for storing electronic instructions. In some embodiments, systems for sample processing comprise a plurality of biological samples collected from distinct or pooled subjects. In some embodiments, the biological samples are nasopharyngeal samples, oropharyngeal samples, oral swab or sponge samples, nasal swab samples, mid-turbinate samples, or saliva samples. In particular embodiments, the biological samples are saliva samples. In other embodiments, the biological samples are NP samples.

In some embodiments, crude biological samples are collected from subjects outside of the system described herein. In some embodiments, crude biological samples are collected and stored in a sample storage container (e.g. tube). The term “crude biological sample” as used herein refers to the biological sample alone, without any additional buffers, reagents, preservatives, etc. For example, a crude biological sample may be saliva. For example, a subject may spit into a sample storage container. As another example, a crude biological sample may be a nasopharyngeal swab. For example, the subject may swab their own nasopharyngeal cavity or have a swab performed by a third party, and the swab containing the nasopharyngeal sample may be placed in a sample storage container. For samples collected using a swab or a sponge, the swab or sponge may be placed in the sample storage container subsequently removed after a suitable amount of time has passed for the sample (e.g. nasopharyngeal sample) to collect within the tube. Alternatively, the swab or sponge may remain within the sample storage container. In some embodiments, the sample storage container comprises a storage buffer. For example, suitable storage buffers include universal transport medium (UTM), phosphate buffered saline (PBS), saline, and the like. The sample storage container may subsequently be brought into/delivered to a system described herein.

In some embodiments, collection supply systems are provided at convenient locations in public or private locations to facilitate ease of access for many different subjects in many different locations. In some embodiments, collection supply systems contain collection supplies such as sample storage containers and instructions for how to use the containers. In some embodiments, collection supply systems contain a zone for collecting, storying, preserving, and/or treating sample storage containers that contain a collected sample. In some such embodiments, the collection supply systems comprise: a first zone containing empty collection supplies (e.g. sample storage containers) that are accessible by subjects that wish to be tested, and a second zone for receiving containers that contain a sample. In some embodiments, the second zone is not accessible by subjects. In some embodiments, the second zone is designed for containing biohazardous materials. In some embodiments, the second zone comprises a pathogen disabling component that disables pathogens present in the second zone (e.g. present in a sample contained in a sample storage container contained in the second zone). In some embodiments, the disabling component comprises a heater that heats the sample(s) to a sufficient temperature for a sufficient time to cause pathogenic agents to no longer present a threat. In some embodiments, the disabling component comprises a source of ultraviolet light that causes pathogenic agents to no longer present a threat. In some embodiments, the second zone is removable from the system to permit transfer of sample storage containers therein to a self-container sample processing system.

In some embodiments, the collection supply system is a box or other container. In some such embodiments, the box may appear similar to a mail collection box to allow it to be readily positioned on a public sidewalk or other public or private area. The box may comprise a first access panel (e.g. door) giving access to the first zone. The box may comprise a second access panel (e.g. door) for depositing sample collection containers so that the collected samples are transferred to the second zone. The second access panel may open to a chute or other pathway that separates the second access panel from the second zone to provide special isolation between the second access panel and the second zone to prevent users from accessing materials collected in the second zone.

Any type of container may be used that is suitable for receiving a sample and storing the sample until processing. Examples of suitable sample storage containers include, but are not limited to, tubes containing a reversibly removal cap, bags, syringes, droppers, and the like. In some embodiments, where saliva is used as the collected sample, the container may contain a funnel component to facilitate transfer of a larger surface area of sample into a smaller container opening (e.g. tube opening). In some embodiments, where a swab is used to collect the sample, the container may be elongated to receive the full swab or may include a cutting component to sever the tip of the swab from a handle and to collect and store the tip containing the collected sample.

Individual sample storage containers may contain a unique barcode or other unique identification indicia. In some embodiments, a user may scan or image the barcode or other indicia with an imaging device (e.g. phone) and have software associated with the imaging device (e.g. an app) associate the identity of the user with the collected sample. In some embodiments, the collection supply system contains a sensor that also reads the barcode or other indicia to identify that that particular sample is housed in that particular collection supply system. A time and/or date stamp may also be associated with the receipt of the sample to assist in determining the time period from sample collection to subsequent sample processing steps (e.g. sample analysis, data collection, data reporting, etc.). In some embodiments, the second zone comprises a removal container that itself has a barcode or other indicia so that batches of samples collected at a particular location can be tracked through the process.

Samples collected at a remote location are transferred to a system described herein for assaying the sample for the presence of an analyte (e.g. pathogen or pathogen component). In some embodiments, the system is a mobile laboratory. In some embodiments, the system is provided in a vehicle, thereby providing a mobile laboratory. In some embodiments, the vehicle is driven or sent to a location of collected samples. For example, in some embodiments, when a collection supply system is sufficiently full (e.g. as determined by a sensor, counter, etc.) a signal is transmitted indicating the same and the vehicle moves to the location of the samples for collection and subsequent processing. In other embodiments, the system is housed on or within a moveable structure, such as a cart, thereby providing a mobile laboratory for use in processing biological samples. The moveable structure (e.g. cart) may be placed in a desired location for a suitable duration of time, and subsequently be moved to a new location as needed. For example, the moveable structure housing the system (e.g. mobile laboratory) may be placed in a building such as a hospital, physicians' clinic, urgent care facility, office space, etc. As another example, the mobile laboratory may be placed in a semi-permanent structure, such as a tent. For example, the mobile laboratory may be placed in a semi-permanent structure which has been erected in an area of high need for sample processing (e.g. an area experiencing high rate or a high potential rate of infection with a pathogen). In general, it may be desirable to move the mobile laboratory from one location to another within 6 months (e.g. within 6 months, within 5 months, within 4 months, within 3 months, within 11 weeks, within 10 weeks, within 9 weeks, within 8 weeks, within 7 weeks, within 6 weeks, within 5 weeks, within 4 weeks, within 3 weeks, within 2 weeks, within 1 week). However, other suitable durations of time may be employed depending on the sample processing needs at a given location.

In some embodiments, the system may be housed in a vehicle (e.g. truck, van, bus, train, plane, etc.). In some embodiments, an operator or rider of a vehicle (e.g. human driver or robot) physically collects samples from a remote location and transfers the samples to a receiving station of the system. The receiving station may be a sample processing station where the initial steps of sample processing occur. Alternatively, the receiving station may be a cue holding sample until ready for processing. In some embodiments, the receiving station comprises one or more sensors to ensure that the samples are positioned correctly for use in subsequent stages of processing within the system. In some embodiments, the receiving station comprises a reader for reading barcodes or other indicia associated with each sample so that the identity of the subject that provided the sample is associated with the data generated from the sample.

The biological samples (e.g. the sample storage tubes containing the samples) may be pre-treated prior to processing. In some embodiments, the samples may be pre-treated in an area outside of the system described herein. In other embodiments, the samples may be pre-treated within the system described herein. For example, samples may be collected from subjects outside of the system, brought into the system (e.g. within sample storage tubes), and subsequently pre-treated within the system. In some embodiments, the biological samples may be pre-treated to inactivate potential pathogens (e.g. virus, bacteria) within the sample. In some embodiments, the biological samples may be pre-treated to lyse cells within the sample, thus releasing the target (e.g. nucleic acid) for subsequent detection. In such embodiments, a pre-treatment step accomplishes both cell or virion lysis (e.g. release of nucleic acid) and inactivation of potential pathogens within the biological sample. In some embodiments, the biological samples may be pre-treated by freezing, heating and/or the addition of a denaturant to the sample. For example, the biological sample may be pre-treated by heating to a sufficient temperature for a suitable duration of time to inactivate potential pathogens within the sample. For example, the biological sample may be heated to about 40° C. or higher. For example, the biological sample may be heated to about 40° C., 45° C., 50° C., 55° C., 60° C., 65° C., 70° C., 75° C., 80° C., 85° C., 90° C., 95° C., 100° C., or more than 100° C. The sample may be maintained at the heated temperature for a suitable duration of time, such as 5 minutes, 10 minutes, 20 minutes, 30 minutes, 40 minutes, 50 minutes, 60 minutes, or more than 1 hour. In particular embodiments, the sample may be heated to 98° C.-100° C. for 5 minutes to accomplish both cell lysis and viral inactivation in a single heat treatment step. In some embodiments, pre-treating the sample comprises adding a denaturant to inactivate potential pathogens within the sample. For example, suitable denaturants include guanidine-based denaturants (e.g. guanidine hydrochloride, guanidine thiocyanate, etc.) and surfactants (e.g., Triton X-100, tween20). In some embodiments, the biological sample does not contain a denaturant. For example, in some embodiments the biological sample (e.g. saliva sample) may not contain a guanidine-based denaturant. In some embodiments, the biological sample (e.g. saliva sample) contains less than 0.3M of a guanidine-based denaturant. For example, the biological sample (e.g. saliva sample) may contain less than 0.3M, less than 0.25M, less than 0.2M, less than 0.15M, less than 0.1M, or less than 0.5M of a guanidine-based denaturant.

The viscosity of certain biological samples (e.g. saliva) makes sample handling difficult. Moreover, the viscosity samples collected from different individuals varies, introducing potential issues with variability of sample collection between subjects. For example, a saliva sample with high viscosity may result in less volume of saliva successfully being pipetted into a desired container (e.g. for subsequent detection of a pathogen in the sample) compared to saliva with decreased viscosity. This introduces potential downstream issues for inaccurate results, including false negative results. In some embodiments, the biological samples may be pre-treated to reduce viscosity of the sample and thereby improve sample handling in subsequent processing steps. In particular embodiments, the pre-treatment step may be performed to inactivate pathogen(s) within the sample and reduce the viscosity of the sample in one step. In some embodiments, one or more agents to decrease viscosity may be added to the biological sample prior to aspirating the sample into the pipette tip. In some embodiments, the agent to decrease viscosity is a reducing agent. Suitable reducing agents include, for example, dithiothreitol (DTT), tris(2-carboxyethyl)phosphine (TCEP), or 2-mercaptoethanol.

Any suitable amount of a reducing agent may be added to the biological sample (or present in the storage buffer in which the biological sample is placed upon collection). In some embodiments, suitable concentrations of reducing agents may range from 0-500 mM. For example, suitable concentrations of DTT or TCEP may range from 0-250 mM (e.g. 0 mM, about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, or about 250 mM). For example, dithiothreitol (DTT)) may be added to a biological sample (e.g. a saliva sample) at a suitable concentration to decrease viscosity of the sample. In some embodiments, DTT may be added to achieve a 1× concentration within the saliva sample. As another example, suitable concentrations of 2-mercaptoethanol may range from 0-500 mM (e.g. 0 mM, about 25 mM, about 50 mM, about 75 mM, about 100 mM, about 125 mM, about 150 mM, about 175 mM, about 200 mM, about 225 mM, about 250 mM, about 275 mM, 300 mM, about 325 mM, about 350 mM, about 375 mM, about 400 mM, about 425 mM, about 450 mM, about 475 mM, or about 500 mM. For example, dithiothreitol (DTT)) may be added to a biological sample (e.g. a saliva sample) at a suitable concentration to decrease viscosity of the sample. In some embodiments, DTT may be added to achieve a 1× concentration within the saliva sample.

In some embodiments, the viscosity reducing agent (e.g. DTT) is added to the biological sample prior to heating the sample (e.g. to inactivate pathogens and/or induce cell lysis). In some embodiments, the viscosity reducing agent is added to the biological sample after heating the sample. In some embodiments, freezing the sample may be performed to reduce the viscosity of the sample. Any suitable pre-treatment step or combination of pre-treatment steps may be performed to achieve the desired result (e.g. cell lysis, pathogen inactivation, and/or reduction of viscosity of the sample).

The biological sample may additionally comprise a suitable detergent. For example, the biological sample may comprise an ionic detergent (e.g. sodium dodecyl sulfate, deoxycholate, cholate, etc.), a non-ionic detergent (e.g. Triton X-100, DDM, digitonin, Tween 20, Tween 40, Pluronic F-127), a zwitterionic detergent, or a chaotropic detergent. In some embodiments, biological sample comprises 0-5% detergent (v/v). For example, the biological sample may comprise 0%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, or about 5% detergent. The detergent may be added to the biological sample or present in a sample storage buffer to which the biological sample is added upon collection.

In some embodiments, the biological samples comprises a non-ionic detergent (e.g. Triton X-100). For example, the biological sample may comprise 0.001-0.1% Triton X-100. The biological sample may be brought to a suitable volume for subsequent use by the addition of a suitable buffer. For example, the biological sample may be brought to a suitable volume by the addition of phosphate buffered saline (PB S), universal transport medium (UTM), saline, and the like. The biological sample may comprise one or more enzymes to assist with breaking down the contents therein to facilitate release of the desired target. For example, the biological sample may comprise one or more enzymes, such as one or more proteases. In particular embodiments, the biological sample may comprise proteinase K. The biological sample may additionally comprise one or more suitable reagents to prevent degradation of the target within the sample. For example, suitable buffers and/or inhibitors (e.g. RNase inhibitors, nuclease inhibitors, etc.) may be added to the biological sample prior to isolating and detecting the target.

The system may additionally comprise one or more heaters, refrigerators, freezers, etc. to ensure appropriate temperature control for samples and/or to perform one or more processing steps. In some embodiments, the system may additionally comprise one or more heaters for sample pre-treatment. In some embodiments, the system may comprise one or more freezers. In some embodiments, the mobile laboratory (e.g. van or cart) may be placed within or moved to a location that contains the heaters, refrigerators, freezers, etc. For example, the mobile laboratory may be placed in or located next to or near a building or semi-permanent structure that contains suitable equipment to heat and/or freeze samples.

It is understood that the methods and processes described herein may be automated (e.g. performed robotically) or may be performed manually. The term “robotically” as used herein in reference to a robotic system (e.g. a robotic sample processing system and/or robotic sample analysis system) refers to a system wherein at least one step in a process performed by that system is performed by a robotic. The term “robotic” does not exclude the possibility that one or more steps may still be performed manually (e.g. by a human). In some embodiments, it may be advantageous for methods to be automated in order to minimize or eliminate human interaction with samples. In other embodiments, it may be advantageous or desirable for one or more steps or processes to be performed manually.

In some embodiments, the systems further comprise a robotic sample processing system. In some embodiments, the robotic sample processing system is configured to (i) mix each biological sample with paramagnetic particles (PMPs) or other capture particles to generate a plurality of compositions, wherein each composition comprises one or more target-PMP complexes, for example, and (ii) isolate the target-PMP complexes from each composition. In some embodiments, the robotic sample processing system comprises a multichannel pipette and an apparatus for operating the multichannel pipette.

In some embodiments, the apparatus for operating the multichannel pipette is configured to induce vertical and/or horizontal movement of the multichannel pipette. For example, the apparatus may comprise a horizontal rod that permits the multichannel pipette to slide in a planar fashion along the length of the rod. In such embodiments, the apparatus for operating the multichannel pipette facilitates movement of the multichannel pipette horizontally along the length of the rod. In some embodiments, the apparatus for operating the multichannel pipette comprises a vertical rod, or an expandable member attached to the multichannel pipette that expands and contracts, thus permitting vertical movement of the multichannel pipette. For example, the apparatus may comprise a vertical rod that permits the multi-channel pipette to slide in a vertical fashion along the length of the rod. In such embodiments, the apparatus for operating the multichannel pipette facilitates movement of the multichannel pipette up or down. Such up and down movement of the multichannel pipette may facilitate attachment of pipette tips thereto, and may facilitate proper placement of the pipette tips contained on the multichannel pipette within a container (e.g. placement within a multi-well plate, placement within a sample collection device, and the like). In some embodiments, the apparatus for operating the multichannel pipette is configured to aspirate and/or inject liquid when pipette tips are attached to the multichannel pipette. The apparatus for operating the multichannel pipette may be controlled by a computer.

In some embodiments, processing the plurality of biological samples comprises mixing each biological sample with capture particles, e.g., PMPs. PMPs or other capture particles may be in a liquid composition or may be in a lyophilized formulation. In some embodiments, processing the plurality of biological samples comprising mixing each biological sample with paramagnetic particles (PMPs) to generate a plurality of compositions, each composition comprising one or more target-PMP complexes. In some embodiments, mixing each biological sample with PMPs is performed in a mixing container. In some embodiments, mixing each biological sample with PMPs or other capture particles is performed in a multi-well plate using a plurality of pipette tips attached to the multichannel pipette. In such embodiments, each biological sample is contained within a distinct well on the multi-well plate. In some embodiments, each well in a multi-well plate (e.g. mixing container) may contain a liquid composition comprising PMPs or other capture particles. Alternatively, each well in a multi-well plate (e.g. mixing container) may contain lyophilized PMPs.

Mixing the biological sample with PMPs, for example, allows the PMPs to bind to the target within the sample, thus generating one or more target-PMP complexes. Target-PMP complexes are subsequently isolated and the presence or absence of a target may be determined. Any suitable paramagnetic particle may be used as the capture particle. In some embodiments, paramagnetic particles may be purchased from a commercial vendor. The specific type of paramagnetic particle used depends on the target to be isolated from the biological sample. For example, particles with a relatively large surface area may be preferable for binding nucleic acid, such as viral RNA. In some embodiments, the paramagnetic particles may be functionalized to aid in capture/purification of the target. For example, the paramagnetic particles may be functionalized with one or more antibodies, aptamers, or other suitable agents to assist with capture of a target. In some embodiments, the paramagnetic particles may be functionalized with one or more protein antibodies to assist with the capture of SARS, coronavirus, SARS-CoV-2 and related targets.

Numerous particles are available that be selected for use in the methods and systems of the invention. See, for example, the particles described in “Magbeads 101: A guide to choosing and using magnetic beads,” which notes various useful capture particles that may be modified, coated, blocked or conjugated for specificity or functionality (https://www.cytivalifesciences.com/en/us/news-center/magnetic-beads-a-simple-guide-10001).

In some embodiments, the liquid composition comprising PMPs or other capture particles contains other suitable reagents for processing/handling of biological samples. For example, the liquid composition comprising PMPs or other capture particles may contain one or more detergents, reducing agents, buffers, inhibitors, enzymes (e.g. proteases), denaturants, etc. Any additional reagents (such as those described above) present in the biological sample may additionally be present in the liquid composition comprising PMPs. For example, the liquid composition may further comprise one or more reagents to decrease viscosity of the biological sample. For example, the liquid composition may comprise PMPs and DTT. The liquid composition may comprise other suitable buffers, inhibitors, and the like to prevent degradation of the target (e.g. target nucleic acid, target protein, etc.) during sample processing. Suitable inhibitors that may be present in the liquid composition comprising PMPs include, for example, RNase inhibitors, protease inhibitors, nuclease inhibitors, and the like. Lyophilized PMP formulations may contain other suitable reagents commonly used in the lyophilization process, including bulking agents, stabilizers, and other suitable excipients.

In some embodiments, the liquid composition comprising PMPs and/or other capture particles may additionally comprise a suitable detergent. For example, the composition may comprise an ionic detergent (e.g. sodium dodecyl sulfate, deoxycholate, cholate, etc.), a non-ionic detergent (e.g. Triton X-100, DDM, digitonin, Tween), a zwitterionic detergent, or a chaotropic detergent. In some embodiments, the composition comprises a non-ionic detergent (e.g. Triton X-100). For example, the composition may comprise 0.001-0.1% Triton X-100.

In some embodiments, the liquid composition may further comprise one or more reagents to decrease viscosity of the biological sample. For example, the liquid composition may comprise PMPs and dithiothreitol (DTT). The liquid composition may comprise other suitable buffers, inhibitors, and the like to prevent degradation of the target (e.g. target nucleic acid, target protein, etc.) during sample processing. The liquid composition may be brought to a suitable volume for subsequent use by the addition of a suitable buffer. For example, the composition may be brought to a suitable volume by the addition of phosphate buffered saline (PBS), saline, and the like.

Any suitable volume of the biological sample may be mixed with the PMPs. For example, about 1 μL to about 500 μL, of the biological sample may be mixed with the PMPs. In some embodiments, a suitable volume of biological sample containing DTT (or another agent to reduce viscosity of the sample) is mixed with the PMPs. For example, about 1-500 μL, of a saliva sample containing DTT may be mixed with the PMPs. For example, 1 μL, 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, or 500 μL of a saliva sample may be used.

Any suitable amount of PMPs or other capture particles may be mixed with one or more biological samples. In embodiments where the capture particles (e.g. PMPs) are contained in a liquid, any suitable volume of the liquid composition comprising paramagnetic particles may be mixed with the biological sample. In some embodiments, the volume of the liquid composition comprising PMPs may equal or exceed the volume of the biological sample. For example, the volume of the liquid composition comprising PMPs may be at least 100%, at least 150%, at least 200%, at least 250%, at least 300%, at least 350%, at least 400%, at least 450%, or at least 500% the volume of the biological sample.

Any suitable concentration of PMPs or other capture particles may be used to ensure sufficient binding of the target capture particles to the target (e.g. formation of a sufficient number of target-PMP complexes). For lyophilized PMP formulations, for example, any suitable weight of lyophilized product may be used to ensure the proper concentration of PMPs to be mixed with the biological sample. For liquid formulations, the liquid composition comprising the PMPs may comprise any suitable concentration of PMPs to ensure sufficient binding of the PMPs to the target (e.g. formation of a sufficient number of target-PMP complexes). For example, PMPs may be present in the liquid composition at about 1-20% (v/v). For example, PMPs may be present in the liquid composition in an amount of about 1%, 2%, 3%, 4%, 5%, 6%, 7%, 8%, 9%, 10%, 11%, 12%, 13%, 14%, 15%, 16%, 17%, 18%, 19%, or about 20% (v/v).

Processing the biological sample further comprises isolating the target-capture particle complexes (e.g. target-PMP complexes generated as a result of mixing the biological sample with PMPs) from each composition. The systems described herein may be used for isolation of any desired target from the sample. For example, the target may be a nucleic acid (e.g. DNA, RNA, or various subtypes thereof including mRNA) or a protein or peptide. For example, the target may be DNA or RNA. In some embodiments, the target may be nucleic acid or proteins or peptides (e.g. antibodies) resulting from a pathogen infecting the subject from which the biological sample was obtained. For example, the target may be bacterial nucleic acid (e.g. bacterial DNA) or viral nucleic acid (e.g. viral RNA). As another example, the target may be antibodies produced by the subject in response to infection with the pathogen.

In some embodiments, isolating target-PMP complexes, for example, comprises aspirating each composition containing the one or more target-PMP complexes into a distinct pipette tip on a multichannel pipette, and positioning the multichannel pipette proximal to a magnet positioned below a sample collection device. In such embodiments, the magnet draws the target-PMP complexes out of the pipette tips and into the sample collection device. In some embodiments, the magnet and/or sample collection device are housed on a moveable surface to facilitate alignment of the sample collection device with the pipette tips on the multichannel pipette.

In some embodiments, the methods described herein comprise aspirating the biological sample into a pipette tip (or other liquid-holding compartment). In some embodiments, the pipette tip may already contain reagents prior to aspiration of the biological sample. Reagents could be in liquid or dry/lyophilized format (e.g., lyophilized magnetic beads functionalized for a target). In some embodiments, the pipette tip contains lyophilized paramagnetic particles (PMPs). Accordingly, aspirating the biological sample into the pipette tip containing the lyophilized paramagnetic particles may initiate mixing the biological sample with the PMPs, thus generating the target-PMP complexes.

In some embodiments, separating or isolating target-PMP complexes from each composition comprises aspirating each biological sample into a pipette tip containing PMPs (e.g. lyophilized PMPs). In some embodiments, isolating target-PMP complexes from each composition comprises mixing each biological sample with PMPs to generate a composition comprising one or more target-PMP complexes, and aspirating each composition into a distinct pipette tip, for example.

In some embodiments, the biological sample or the composition containing the one or more target-PMP complexes is aspirated through a bottom opening of a pipette tip. In other embodiments, the biological sample or the composition containing the one or more target-PMP complexes is aspirated through an opening on the side of the pipette tip (e.g. a side opening). For example, the pipette tip may contain a bottom opening and a side opening, and the bottom of the pipette tip may be placed against a surface such that the pipette tip and the surface have conformal contact (e.g., the bottom of the pipette tip is flush against the surface) and no liquid is able to enter the pipette tip through the bottom opening. Once the appropriate contact between the bottom opening and the surface is achieved, liquid may be aspirated through the side opening of the pipette tip. Such a method will generate a pocket of air proximal to the bottom opening of the pipette tip during aspiration of liquid through the side opening.

In some embodiments, isolating target-PMP complexes from each composition comprises generating a liquid/air interface, a liquid/oil interface, or a hybrid interface proximal to a bottom opening of the pipette tip. This step may be performed after aspirating the biological sample or the composition comprising one or more target-PMP complexes, such as after aspirating the biological sample or composition through a bottom opening of the pipette tip. In some embodiments, separating or isolating target-PMP complexes comprises aspirating each biological sample or composition comprising the one or more target-PMP complexes into a distinct pipette tip through a bottom opening of the pipette tip, and subsequently generating a liquid/air interface, a liquid/oil interface, or a hybrid interface proximal to the bottom opening of the pipette tip. Alternatively, generating the liquid/air interface, liquid/oil interface, or hybrid interface may occur during aspiration of the biological sample or composition comprising the one or more target-PMP complexes, such as during aspiration through a side opening of the pipette tip.

In some embodiments, the apparatus for operating the multichannel pipette is configured to create the interface at the bottom opening of the plurality of pipettes attached thereto. For example, the apparatus may be configured to further aspirate the composition following formation of target-capture particle complexes (e.g. after mixing the biological sample with PMPs) within the pipette tips while the bottom opening of the pipette tip is exposed to air. Such further aspiration will create a suitable interface between the meniscus of the liquid contained within the pipette tip and the pocket of air and/or oil (or another suitable hydrophobic material) at the bottom opening.

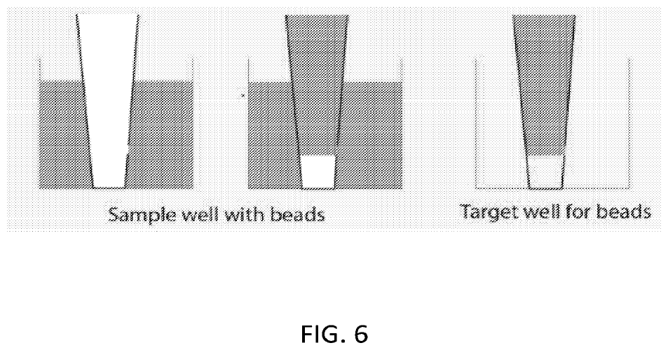

The liquid/air interface is an interface between the composition contained within a pipette tip or other useful liquid-holding compartment (e.g. the biological sample mixed with PMPs), and the air. The liquid/air interface may be generated by further aspirating the composition within a pipette tip, for example, while the pipette tip is exposed to the external environment, thus generating a pocket of air at the bottom opening of the pipette tip. Alternatively, the liquid/air interface may be generated during aspiration of the composition or the biological sample through a side opening of the pipette tip, as described above and as shown in FIG. 6.

The term “liquid/oil interface” is used herein to broadly refer to an interface between the composition contained within the pipette tip and a hydrophobic substance. Although the hydrophobic substance is commonly referred to herein as oil, it is understood that other suitable hydrophobic substances may be used. In some embodiments, the hydrophobic substance is an oil or a hydrophobic polymer. In some embodiments, the hydrophobic substance is a wax. In some embodiments, the liquid/oil interface may be generated by dipping the pipette tip into a suitable oil (e.g. light mineral oil) and further aspirating the composition, thus generating a pocket of oil at the bottom opening of the pipette tip.

In some embodiments, the methods comprise generating a hybrid interface proximal to the bottom opening of a pipette tip (or other liquid-holding compartment). The hybrid interface may comprise a layer of a hydrophobic substance coating the inner walls of, for example, the pipette tip and a pocket of air proximal to the bottom opening of the pipette tip. The hydrophobic substance may be any suitable substance, including an oil, a wax, or a hydrophobic polymer. In some embodiments, pipette tips are designed and/or purchased containing the layer of the hydrophobic substance (e.g. wax, hydrophobic polymer) already present within the pipette tip. In other embodiments, the hydrophobic substance may be generated within the pipette tip manually or robotically (e.g. using a multichannel pipette as described herein). In some embodiments, the hybrid interface may be generated by dipping the pipette tip into a hydrophobic substance such as an oil (e.g. light mineral oil), pulling the pipette tip out of the hydrophobic substance and into the air, and aspirating a pocket of air into the bottom opening of the pipette tip.

The volume of air aspirated into the oil-coated pipette tip may be about 1 μL to about 20 μL. The volume aspirated may depend on the diameter/size of the pipette, volume of liquid, viscosity of the sample, type of paramagnetic particles used, or a combination thereof. For example, when a p200 pipette tip is used the volume of air aspirated into the oil-coated pipette tip may be about 8-12 μL (e.g. 8 μL, 9 μL, 10 μL, 11 μL, or 12 μL).

In some embodiments, the pocket of air and/or oil generated is 0.25-30 mm high, as measured from the bottom opening of the pipette tip along the side of the tip. For example, the pocket of air may be 0.25 mm, 0.5 mm, 0.75 mm, 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, or 30 mm high. The appropriate height and/or volume of the pocket of air may be selected based on the diameter of the opening of the pipette tip, the temperature of the biological sample, the viscosity of the biological sample, the nature of the capture (e.g. paramagnetic) particles used, the strength of the magnetic force, etc.

In some embodiments, the methods further comprise positioning the multichannel pipette proximal to a first magnet to generate a concentration of target-PMP complexes proximal to the liquid/air interface, liquid/oil interface, or hybrid interface. The first magnet may therefore also be referred to herein as a “preconcentration magnet”. For example, a multichannel pipette containing multiple pipette tips holding the compositions containing the one or more target-PMP complexes may be moved in proximity to a first magnet. Alternatively, a first magnet may be moved in proximity to the multichannel pipette containing the multiple pipette tips. The first magnet will attract the PMPs within the composition, thus pulling target-PMP complexes towards the first magnet. The location of the magnet in relation to the pipette tip will determine the area within the pipette tip at which the target-PMP complexes will collect. Accordingly, the magnet should be appropriately placed relative to the pipette tip to ensure that the target-PMP complexes collect proximal to the liquid/air interface, liquid/oil interface, or hybrid interface.

Following generating the concentration of target-PMP complexes proximal to the liquid/air interface, liquid/oil interface, or hybrid interface at the bottom opening of the pipette tip, the methods further comprise drawing the target-PMP complexes through the interface and into a suitable sample collection device. Accordingly, the methods further comprise positioning the multichannel pipette proximal to a second magnet to draw the target-PMP complexes through the liquid/air interface, liquid/oil interface, or hybrid interface and into a sample collection device. The second magnet may also therefore be referred to herein as a “sample collection magnet”. The second magnet may be a separate magnet from the first magnet. In some embodiments, the second magnet may be stronger than the first magnet. For example, the first magnet may be of sufficient strength to draw target-PMP complexes towards the magnet but not strong enough to pull target-PMP complexes through the liquid/air interface, liquid/oil interface, or hybrid interface. In contrast, the second magnet is of sufficient strength to achieve purification of target-PMP complex by drawing the target-PMP complex through the liquid/air interface, liquid/oil interface, or hybrid interface. Drawing the target-PMP complex through the interface represents a facile means for purifying the target from the biological sample without the need for additional processing steps that may result in sample loss or contamination with unwanted components from the biological sample itself.

The second magnet may be positioned in a suitable location such that the target-PMP complexes are drawn through the liquid/air interface, liquid/oil interface, or hybrid interface and into a sample collection device. For example, the second magnet may be positioned below the sample collection device. In some embodiments, the sample collection device is a multi-well plate. In such embodiments, the second magnet may be positioned below the multi-well plate. The multichannel pipette is positioned above the multi-well plate (e.g. above the second magnet) such that the target-PMP complexes within each pipette tip are drawn into a distinct well on the multi-well plate. In some embodiments, the system comprises a plurality of sample collection magnets (e.g. arranged in an array). For example, a plurality of sample collection magnets may be used to address a plurality of pipette tips and/or pipettes at the same time or in sequence. In some embodiments, a third set of magnets are used to influence or adjust the uniformity and strength of the sample collection magnets. For example, when the sample collection magnets are arranged in an array pattern, a third set of magnets positioned around the perimeter of the array to reduce edge effects, maintaining a more consistent magnetic field for each sample collection magnet in the array. Accordingly, the third set of magnets may be referred to herein as “field stabilization magnets”.

In some embodiments that use pipette tips and PMPs, separating or isolating target-PMP complexes from each biological sample comprises:
a. Aspirating each composition into a distinct pipette tip; and
b. Positioning the multichannel pipette proximal to a magnet positioned below a sample collection device, such that the magnet draws the target-PMP complexes out of the pipette tips and into the sample collection device.

In some embodiments that use pipette tips and PMPs, separating or isolating target-PMP complexes from each composition comprises:
a. Aspirating each biological sample into a distinct pipette tip containing lyophilized PMPs to generate a composition comprising one or more target-PMP complexes within each pipette tip; and
b. Positioning the multichannel pipette proximal to a magnet positioned below a sample collection device, such that the magnet draws the target-PMP complexes out of the pipette tips and into the sample collection device.

In some embodiments with pipette tips and PMPs, separating or isolating the target-PMP complexes from each composition comprises:
a. Aspirating each composition into a distinct pipette tip;
b. Generating a liquid/air interface, a liquid/oil interface, or a hybrid interface proximal to a bottom opening of the pipette tip; and
c. Positioning the multichannel pipette proximal to a magnet positioned below a sample collection device, such that the magnet draws the target-PMP complexes through the liquid/air interface, liquid/oil interface, or hybrid interface and into the sample collection device.

In other embodiments, separating or isolating target-PMP complexes from each composition comprises:
a. Aspirating each composition into a distinct pipette tip;
b. Generating a liquid/air interface, a liquid/oil interface, or a hybrid interface proximal to a bottom opening of the pipette tip;
c. Positioning the multichannel pipette proximal to a first magnet to generate a concentration of target-PMP complexes proximal to the liquid/air interface, liquid/oil interface, or hybrid interface; and
d. Positioning the multichannel pipette proximal to a second magnet to draw the target-PMP complexes through the liquid/air interface, liquid/oil interface, or hybrid interface and into a sample collection device.

In some embodiments that use pipette tips and PMPs, separating or isolating the target-PMP complexes from each composition comprises:
a. Aspirating each biological sample into a distinct pipette tip containing lyophilized PMPs to generate a composition comprising one or more-target PMP complexes within each pipette tip;
b. Generating a liquid/air interface, a liquid/oil interface, or a hybrid interface proximal to a bottom opening of the pipette tip; and
c. Positioning the multichannel pipette proximal to a magnet positioned below a sample collection device, such that the magnet draws the target-PMP complexes through the liquid/air interface, liquid/oil interface, or hybrid interface and into the sample collection device.

In other embodiments with pipette tips and PMPs, separating or isolating the target-PMP complexes from each composition comprises:
a. Aspirating each biological sample into a distinct pipette tip containing lyophilized PMPs to generate a composition comprising one or more-target PMP complexes within each pipette tip;
b. Generating a liquid/air interface, a liquid/oil interface, or a hybrid interface proximal to a bottom opening of the pipette tip;
c. Positioning the multichannel pipette proximal to a first magnet to generate a concentration of target-PMP complexes proximal to the liquid/air interface, liquid/oil interface, or hybrid interface; and
d. Positioning the multichannel pipette proximal to a second magnet to draw the target-PMP complexes through the liquid/air interface, liquid/oil interface, or hybrid interface and into a sample collection device.

In some embodiments, the distinct wells on the multi-well plate comprise a wash buffer. In some embodiments, the wash buffer comprises water. In some embodiments, the wash buffer comprises ethanol. In some embodiments, a layer of oil (e.g. light mineral oil) may reside above the wash buffer. Accordingly, target-capture particle (e.g. PMP) complexes will be drawn through the layer of oil prior to contacting the wash buffer, thus further purifying unwanted contaminants from the target-PMP complexes. In some embodiments, processing the plurality of biological samples further comprises aspirating the wash buffer (and the layer of oil, if present) from the wells and allowing the target-PMP complexes contained therein to dry. In some embodiments, additional wash steps may be performed. In some embodiments for detecting the target may be added to the target-PMP complexes after removal of the wash buffer, and suitable means for detecting the target may be used (e.g. a plate reader, naked-eye visualization, etc.) In some embodiments, the workflow of the system may be optimized such that two plate readers may be used, thus maximizing throughput.

In some embodiments, the system further comprises a moveable surface. One or multiple components of the system may be placed on the moveable surface. The moveable surface may change orientation and/or move in the x-y plane and or in the vertical z-direction. Such movement may facilitate proper positioning of one or more components on the surface relative to the multichannel pipette. For example, magnets (e.g. the first magnet, the second magnet), the biological samples, and/or the sample collection device may be housed on the moveable surface.

In some embodiments, the system comprises a plurality of pipette tips held within a suitable pipette tip container. The pipette tip container may be placed on a moveable surface. For example, the pipette tips may be held in a container placed on a moveable surface, and the moveable surface may be positioned to align the plurality of pipette tips with the multichannel pipette. Following proper alignment, the moveable surface and/or the multichannel pipette may be moved vertically (e.g. up or down) to permit attachment of the pipette tips to the multichannel pipette.

In some embodiments, the system described herein comprises a plurality of biological samples, wherein each biological sample is held within a multi-well plate. The biological samples may be pre-treated by heating, addition of a denaturant, and/or treatment with a viscosity reducing agent, as described above, prior to placing in the multi-well plate. For example, the biological samples may be initially collected into a sample storage tube, pre-treated, and moved into distinct wells on a multi-well plate. The multi-well plate may be housed upon a moveable surface. The moveable surface and/or the multichannel pipette (e.g. containing a plurality of pipette tips) may move in a suitable fashion to facilitate alignment of the pipette tips with the plurality of biological samples. The apparatus for operating the multichannel pipette may aspirate the biological samples, such that each biological sample is aspirated into a different pipette tip.

In some embodiments, the systems described herein further comprise a mixing container for combining and subsequently mixing biological samples with the target capture (e.g. paramagnetic) particles. In some embodiments, the mixing container comprises a multi-well plate. In some embodiments, a plurality of wells in the multi-well plate contain a liquid composition comprising PMPs, for example, such that a plurality of biological samples may be mixed with the plurality of liquid compositions comprising PMPs simultaneously. In other embodiments, a plurality of wells in the multi-well plate contain lyophilized PMPs. In some embodiments, the apparatus for operating the multichannel pipette injects the liquid composition comprising PMPs into each of the plurality of wells prior to mixing the liquid composition with the biological samples. In some embodiments, the mixing container is housed on a moveable surface. In some embodiments, the system apparatus for operating the multichannel pipette and/or the moveable surface are configured to position the multichannel pipette such that the plurality of pipette tips are aligned with a plurality of wells in the mixing container (e.g. multi-well plate), such that the biological samples may be mixed with the PMPs contained therein. The apparatus for operating the multichannel pipette may facilitate aspiration and injection of liquid by the multichannel pipette, such that the biological samples and the PMPs may be mixed. Mixing of the biological samples with the PMPs results in formation of target-PMP complexes.

In some embodiments, the system further comprises a first magnet. The first magnet may be of suitable strength to pull the target-PMP complexes to a desired area within the pipette tip. For example, the first magnet may be of suitable strength to pull the target-PMP complexes towards the bottom of the pipette tip to form a concentration of target-PMP complexes proximal to the bottom opening of the pipette tip. Accordingly, the first magnet may also be referred to as a “preconcentration magnet”. In some embodiments, the first magnet may be placed on a moveable surface. The apparatus for operating the multichannel pipette and/or the moveable surface may position the multichannel pipette appropriately with relation to the first magnet to achieve formation of the concentration of target-PMP complexes in the desired location.

In some embodiments, the system further comprises a sample collection device. For example, the sample collection device may be a multi-well plate. The sample collection device (e.g. multi-well plate) may be housed on a moveable surface. The moveable surface and/or the multichannel pipette may move to align each pipette tip on the multichannel pipette with a separate well in the sample collection device (e.g. multi-well plate).

In some embodiments, the system further comprises a second magnet. In some embodiments, the second magnet is stronger than the first magnet. The second magnet may be of suitable strength to draw target-PMP complexes, for example, through the liquid/air, liquid/oil, or hybrid interface at the bottom opening of each pipette tip and into the sample collection device. Accordingly, the second magnet may also be referred to herein as a “sample collection magnet”. However, the second magnet does not need to be stronger than the first magnet. In some embodiments, proximity may be employed rather than increased strength to effectuate the movement of target-PMP complexes through the interface. For example, the first magnet and the second magnet may be of the same strength, but the second magnet may be placed in closer proximity to the pipette tips in order to draw the target-PMP complexes contained therein through the liquid/air, liquid/oil, or hybrid interface. In some embodiments, the sample collection device is a multi-well plate. The second magnet may be positioned at a suitable location to draw target-PMP complexes into the sample collection device. For example, the second magnet may be positioned below the multi-well plate. In some embodiments, the apparatus for operating the multichannel pipette and/or a moveable surface housing the multi-well plate is configured to position the multichannel pipette above the multi-well plate, such that each pipette tip is aligned with a well in the multi-well plate, thus permitting target-PMP complexes present in each pipette tip to be pulled through the liquid/air interface by the second magnet and into a well.

In some embodiments, the apparatus for operating the multichannel pipette is configured to aspirate and/or inject liquid. In particular embodiments, the apparatus for operating the multichannel pipette is configured to mix the biological samples with a PMPs to generate a composition comprising one or more target-PMP complexes. In some embodiments, the apparatus is further configured to generate a liquid/air, liquid/oil, or hybrid interface proximal to a bottom opening of the pipette tip.

In some embodiments, the apparatus for operating the multichannel pipette is configured to:
a. Mix the biological sample with paramagnetic particles (PMPs), for example, to generate a composition comprising one or more target-PMP complexes within a pipette tip, for example, and
b. Generate a liquid/air interface proximal to a bottom opening of the pipette tip.

In particular embodiments, the apparatus for operating the multichannel pipette is configured to:
a. Mix the biological sample with paramagnetic particles (PMPs), for example, to generate a composition comprising one or more target-PMP complexes,
b. Aspirate the composition into a pipette tip through a bottom opening of the pipette tip; and
c. Generate a liquid/air interface proximal to the bottom opening of the pipette tip.

In particular embodiments, the apparatus for operating the multichannel pipette is configured to:
a. Mix the biological sample with paramagnetic particles (PMPs), for example, to generate a composition comprising one or more target-PMP complexes,
b. Position a pipette tip such that a bottom opening of the pipette tip is in conformal contact with a surface, such that liquid is unable to enter the pipette tip through the bottom opening, and
c. Aspirate the composition into a pipette tip through a side opening of the pipette tip, thereby generating a liquid/air, liquid/oil, or hybrid interface proximal to the bottom opening of the pipette tip.

In particular embodiments, the apparatus for operating the multichannel pipette is configured to perform the following steps, in order:
a. Aspirate biological samples into pipette tips attached to the multichannel pipette;
b. Pipette biological samples into mixing container holding PMPs, for example;
c. Mix the biological samples with the PMPs to generate a composition comprising one or more target-PMP complexes within each pipette tip; and
d. Further aspirate the composition within each pipette tip to generate a liquid/air interface at a bottom opening of each pipette tip.

In some embodiments, the apparatus for operating the multichannel pipette is configured to move the multichannel pipette (e.g. change orientation and/or move in the x-y plane and or in the vertical z-direction). In particular embodiments, the apparatus for operating the multichannel pipette is configured to perform one or more of the following:
a. Position the multichannel pipette above a container holding a plurality of pipette tips;
b. Move the multichannel pipette downward to attach a plurality of pipette tips to the multichannel pipette;
c. Position the multichannel pipette above a mixing container;
d. Move the multichannel pipette downward to permit contact between the plurality of pipette tips and the contents within the mixing container;
e. Position the multichannel pipette proximal to a first magnet;
f. Position the multichannel pipette proximal to a second magnet.

In some embodiments, the apparatus for operating the multichannel pipette is further configured to move the multichannel pipette to a position above a waste basin and eject used pipette tips into the waste basin.

In some embodiments, the systems described herein comprise a robotic sample analysis system. The robotic sample analysis system is configured to receive separated or isolated target-capture particle (e.g. PMP) complexes and carry out an assay, for example, a biological assay to detect the presence or amount of said target, if present. Accordingly, the robotic sample analysis system may comprise the sample collection device, as described herein, along with reagents for detecting and/or quantifying the target and optionally additional equipment necessary for performing an assay for detecting the presence or amount of the target, if present in the biological sample.

The systems described herein additionally comprise reagents for detecting the target following isolation. In some embodiments, the reagents for detecting the target comprise reagents for loop-mediated isothermal amplification (LAMP)-based detection of the presence or amount of the target. In general, LAMP reactions include a DNA polymerase with strong strand displacement activity and tolerance for elevated temperatures and up to six DNA oligonucleotides of a certain architecture. RT-LAMP reactions additionally include a reverse transcriptase. Samples with potential template molecules are added to the reaction and incubated for 20 to 60 min at a constant temperature (e.g., 65° C.). The oligonucleotides act as primers for the reverse transcriptase, and additional oligonucleotides for the DNA polymerase are designed so the DNA products loop back at their ends. These, in turn, serve as self-priming templates for the DNA polymerase. In the presence of a few RNA template molecules, a chain reaction is set in motion, which then runs until the added reagents (in particular, the deoxynucleotide triphosphates) are used up.

In particular embodiments, the reagents are for a colorimetric assay for detecting the target. Such embodiments allow for a facile visualization of whether or not the sample contains the target of interest. In some embodiments, the reagents are for a colorimetric loop mediated isothermal amplification (LAMP) assay. In embodiments wherein the nucleic acid is RNA, the sample collection device may contain reagents for a colorimetric RT-LAMP assay. LAMP assays or RT-LAMP assays may be a preferred embodiment due to their rapid nature, one-tube processing, and easy visualization of results without the need for expensive equipment or additional materials. In some embodiments, the reagents for a colorimetric LAMP assay (or colorimetric RT-LAMP assay) further include an indicator, which permits evaluation of a color change in the sample in the presence of sufficient nucleic acid (e.g. the target nucleic acid which the LAMP or RT-LAMP reagents are designed to detect). Suitable indicators include pH-sensitive indicators and metal-sensitive indicators. In preferred embodiments, pH-sensitive indicators (e.g. phenol red) may be used, due to their easy visualization with the naked eye.

In some embodiments, the reagents for detection of the target comprise reagents for a fluorescent assay for detecting the target. For example, the sample collection device may contain reagents for a fluorescent LAMP or fluorescent RT-LAMP assay. Any suitable fluorescent dye may be used in a fluorescent LAMP or fluorescent RT-LAMP assay to permit a fluorescent signal to be generated in the presence of sufficient nucleic acid.

In some embodiments, the reagents comprise oligonucleotides (e.g. primers) designed for detection of bacterial nucleic acid. In some embodiments, the reagents comprise oligonucleotides designed for detection of viral RNA. For example, the reagents may comprise oligonucleotides designed for detection of a viral upper respiratory infection selected from SARS-CoV2, SARS, a coronavirus, rhinovirus, influenza, respiratory syncytial virus, etc. In some embodiments, the reagents comprise oligonucleotides for detection of SARS-CoV-2 RNA. In particular embodiments, the reagents comprise oligonucleotides designed for detection of SARS-CoV-2 RNA.

Any of a wide variety of other assay methodologies may be employed, including, but not limited to, polymerase chain reaction (PCR), ligase chain reaction (LCR), NASBA, sequencing, thermophilic helicase-dependent amplification (tHDA), transcription-mediated amplification (TMA), strand displacement amplification (SDA), recombinase polymerase amplification (RPA), and the like. Other nucleic assays may be used, e.g, RT-PCR, as may protein detection assays. Other assays that may be used include, for example, ELISAs, RIAs, which may be quantitative, semi-quantitative, or qualitative. Serology assays to detect IgM, IgG or total antibodies may also be used to evaluate past exposure and immune status, for example.

In some embodiments, the reagents are added to the sample collection device, and retained within the sample collection device (e.g. multi-well plate) by a sealant. Suitable sealants include, for example, oils that are liquid at room temperature. For example, the sealant may comprise mineral oil. For example, a layer of mineral oil may float above the reagents for detection of the target.

In some embodiments, the isolated target-PMP complexes are contacted with the reagents for LAMP-based detection of the target, and a signal resulting from contact is measured. For example, the signal may be a colorimetric signal (e.g. a signal from a colorimetric RT-LAMP assay) or a fluorescent signal (e.g. a signal from a fluorescent RT-LAMP assay). In some embodiments, the target-PMP complexes are drawn into a sample collection device containing a wash buffer, the wash buffer is aspirated from the wells while the sample collection device is still in proximity to a magnet, thereby removing the wash buffer while leaving behind the target-PMP complexes. Subsequently, the target-PMP complexes contained therein may be allowed to dry. Following a suitable time for drying, reagents for LAMP-based detection of the target may be added to each well. In some embodiments, the reagents are added manually. In other embodiments, the reagents are added in whole or in part by the robotic sample processing system (e.g. the multi-well pipette). The signal resulting from contacting the target-PMP complexes with the reagents is measured. For example, a colorimetric signal (e.g. a color change) or a fluorescent signal may be measured to determine which wells contain the target. Measuring a signal (e.g. color change, fluorescent signal) may occur, for example, by visualization (e.g. by the naked eye). Alternatively, the signal may be measured using equipment, such as a plate reader. For example, a fluorescent signal may be measured using a plate reader.

In some embodiments, the system comprises a computer. In some embodiments, the system (e.g. mobile laboratory) contains a computer system comprising one or more processors, wherein the processors are configured to: i) catalog a plurality of biological samples collected from distinct subjects; ii) operate a robotic sample processing system; iii) operate a robotic sample analysis system; iv) associated data received from said sample analysis system with individual members of said plurality of biological samples; and v) remotely communicate said data. For example, the apparatus for operating the multichannel pipette may be controlled by a computer. For example, the computer may dictate movement of the multichannel pipette (e.g. movement along a horizontal or vertical rod) and instruct the apparatus for operating the multichannel pipette to induce aspiration/injection of liquid by the multichannel pipette. As another example, the moveable surface may be controlled by a computer. In some embodiments, the apparatus for operating the multichannel pipette and the moveable surface are both controlled by a computer.

The computer may comprise a processor and a memory. The memory may contain software which instructs the processor to execute a given task. For example, the memory may contain software which instructs the processor to cause the apparatus for controlling the multichannel pipette to perform a variety of tasks related to the methods described herein. For example, the memory may contain software which instructs the processor to cause a multichannel pipette to attach pipette tips to the pipette, aspirate a biological sample, mix the biological sample with PMPs to generate a composition comprising one or more target-PMP complexes, position the pipette tips appropriately relative to a first magnet, position pipette tips appropriately relative to a second magnet, and other necessary functions to perform the claimed method. As another example, the memory may contain software which instructs the processor to cause a moveable surface housing one or more components (e.g. sample collection devices, mixing containers, etc.) to move to a desired position.

The systems described herein may further comprise a memory component. The memory may be the same memory described above which contains software which instructs the processor to execute a given task. The memory stores data regarding detection of the target for each of the plurality of biological samples. Data regarding detection of the target may be data related to the signal resulting from contacting the target-PMP complexes with the reagents for LAMP-based detection of the target. For example, data regarding detection of the target may be data related to the colorimetric or fluorescent RT-LAMP signal resulting from contacting the target-PMP complexes containing viral nucleic acid with reagents for RT-LAMP. In some embodiments, the data comprises time to threshold values for the colorimetric signal to reach a predetermined threshold. In other embodiments, the data comprises cycle threshold (CT) values (e.g. the number of cycles required for the signal to reach a predetermined threshold value). In some embodiments, the data regarding detection of the target is correlated with a unique identifier for each biological sample. For example, biological samples obtained from distinct subjects may each be assigned a unique identifier. This unique identifier will be maintained throughout the sample processing steps. For example, the data regarding detection of the target can be traced to a distinct well on the multi-well plate, which can be traced to target-PMP complexes obtained from a distinct biological sample, which can be assigned to a distinct individual.

In some embodiments, the biological sample is obtained from a subject suspected of having an infection. The subject may be suspected of having any infection by a pathogen that can be detected in the saliva or nasopharyngeal sample from the subject, or an infection which causes the subject to produce antibodies which may be detected in saliva or a nasopharyngeal sample from the subject. In some embodiments, the subject may be suspected of having a bacterial infection or a viral infection. For example, the subject may be suspected of having an upper respiratory infection. For example, the subject may be suspected of having a viral upper respiratory infection, including infection with SARS-CoV-2, a coronavirus, rhinovirus, influenza, respiratory syncytial virus, and the like. In such embodiments, the data regarding detection of the target is informative of whether or not the subject actually has the infection. For example, if the target is SARS-CoV2 RNA, the data regarding detection of the SARS-CoV2 RNA may be used to determine whether or not the subject is infected with SARS-CoV2.

The systems described herein may be used in conjunction with a suitable means for collecting samples from a plurality of subjects. For example, a subject may obtain a sample storage tube by a suitable means, provide their biological sample into the sample storage tube, and the sample storage tube may be collected and placed within the system described herein for downstream processing. For example, subjects may request a sample storage tube, which can be delivered to the subject by a suitable means (e.g. delivered by mail, placed at a location for the subject to pick it up). The subject may provide background information (e.g. name, address, age, date of birth, etc.) which may be used to assign the sample storage tube with a unique identifier affiliated with the subject. The subject may provide their biological sample into the sample storage tube (e.g. provide a saliva sample by spitting into the tube). The sample storage tube container the unique identifier affiliated with that subject may be placed at a sample drop-off location. The sample drop-off location may be, for example, a box capable of containing multiple samples. The box may be equipped with a sensor to verify when a sample is placed within, and/or a sensor to count the number of samples within. Once a sufficient number of samples have been collected, the box could alert the system that samples are ready for collection. A mobile system could then pick up the samples contained within the box. The samples may then be processed within the system as described herein.

In some embodiments, biological samples may be pooled and subsequently used in the methods described herein. For example, biological samples may be collected from a plurality of distinct individuals, pooled together, and used in the methods described herein to determine whether a population has cases of infection with a pathogen (e.g. with SARS-CoV2). As another example, a plurality of biological samples may be collected from an individual, and the plurality of biological samples from a distinct individual may be pooled to increase the amount of sample available to be used in the methods described herein. Such embodiments may be useful for instances where an individual may have a low viral load, or when multiple tests may be performed using the same sample.

In some embodiments, a single pipette tip (e.g. a pipette tip containing a sample collected from one individual) may be used such that a composition contained therein can be subjected to the magnet-based purification process described herein multiple times. For example, a composition contained within a pipette tip (such as a biological sample obtained from a single individual) may be placed above magnet, wherein the magnet is placed below a sample collection device such that a portion of the target-PMP complexes contained therein are drawn into the sample collection device. The pipette tip and/or sample collection device may be moved, and a second portion of the target-PMP complexes contained therein may be drawn into a second sample collection device. For example, the sample collection device may be a multi-well plate. The first portion of the target-PMP complexes may be drawn into a first well, the second portion may be drawn into a second well, a third portion may be drawn into a third well, etc. until the desired number of distinct wells containing target-PMP complexes are obtained. In this manner, the assay may be multiplexed such that a single composition provides sufficient target-PMP complexes for performing multiple target detection assays.

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and as practical applications of the disclosure to enable one skilled in the art to utilize the disclosure in various embodiments and with various modifications as suited to the particular use contemplated. It is intended that the scope of the disclosure be defined by the claims appended hereto and their equivalents.

### EXAMPLES

### Example 1

### Reagents, Standards, Supplies, and Equipment

### Reagents and Standards:

The assay used for this work was the NEB WarmStart LAMP Kit (DNA & RNA)—NEB catalog E1700. Store at −15 to −25° C. through the kit expiration date. Avoid repeated freezing and thawing. Kits included the following reagents:
i. WarmStart LAMP 2× Master Mix
ii. Fluorescent dye (50×)

20× stock of primers specific to SARS-CoV-2 Genes N and As1e—Lyophilized primers were purchased from IDT or Biosynthesis and reconstituted with nuclease free ddH₂O to a final concentration of 100 μM. Stored at −15 to −25° C. through the expiration date. Primers were then mixed to a final concentration of 2.5×:
i. F3—1 μM
ii. B3—1 μM
iii. FIP—8404
iv. BIP—84 μM
v. LF—2 μM
vi. LB—2 μM

20× stock of primers specific to RNAseP—Lyophilized primers were purchased from IDT or Biosynthesis and reconstituted with nuclease free ddH2O to a final concentration of 100 μM. Stored at −15 to −25° C. through the expiration date. Primers were then mixed to a final concentration of 10×:
vii. F3—2 μM
iii. B3—2 μM
iv. FIP—16 μM
v. BIP—16 μM
vi. LF—4 μM
vii. LB—4 μM

SeraSil-Mag Silica Coated Superparamagnetic Bead Mixture (Cytiva)—Mixed thoroughly before use. Stored at ambient temperature.

Bleach, minimum 5% or 0.7M sodium hypochlorite solution

Phosphate-Buffered Saline (PBS) pH 7.4—Gibco catalog #10010-023 or equivalent. Stored at ambient temperature.

Sample Buffer—1M DTT and 1% Triton x-100 in PBS (pH 7.4) Stored at ambient temperature.

RNase-Free, DNase-Free Water—Invitrogen catalog AM9937(or equivalent). Stored at ambient temperature.

Light Mineral Oil—Millipore Sigma catalog #330779 or equivalent. Stored at ambient temperature.

SARS-CoV-2 Positive controls—Prepared aliquots of a diluted inactivated (heat or irradiation) SARS-Cov-2 virion in 1M NaCl PBS (pH 7.4). RNAseP Positive control—Prepared aliquots of a diluted human genomic DNA in PBS (pH 7.4). Positive and negative controls consist of a viral load at 1.0×10⁴ for SARS-CoV-2, 2.0 ng/uL for RNAseP, and 0 copies/mL. Stored at ≤−80° C. and ambient temperatures

### Supplies

Personal Protective Equipment (PPE): Latex free Nitrile gloves, pre-amplification lab coat and safety glasses

10% (v/v) bleach (0.5% (w/v) sodium hypochlorite) made fresh daily and 70% ethanol Biohazard disposal containers (for sharps and non-sharps)

1.5 mL polypropylene screw cap tubes, sterile, non-siliconized, and conical (Sarstedt 72.693.105 or equivalent)

Sterile disposable serological pipets, 10 and 25 mL

Sterile fine-tip transfer pipettes, RNase-free

Wypalls

Pipette tips (capacity 1000 μL, 200 μL, 20 μL, 10 μL) plugged (aerosol barrier). Plugged (aerosol barrier) tips are used to prevent reagent and amplicon cross contamination

RNAse Away, Fisher 14-375-35, 11, Store at ambient temperature.

Equipment

Pre Amplification Area
Gilson PipetMax liquid handling robot was used, an automated pipetting solution useful for the efficient processing of high-throughput biological assays, but other automated liquid handling systems may be used, including systems from Tecan, Hamilton, etc. Robot arms from Omron or other manufacturers may be used in the systems.
On stage magnetic arrays for PipetMax
On stage tip box holders for PipetMax
Networked Personal Computer with Trilution 3.0, R studio programs for managing Pipetmax workflows, and other software that, among other functions, is designed for use in controlling the communication between components, routing data to the cloud, etc., and peripherals
Pipettes (capacity 1000 μL, 200 μL, 20 μL, 10 μL, multi and single channel). Pipettes should be accurate within 3% of stated volume.
−20° C. Reagent Freezer
−80° C. Specimen Freezer
2-8° C. Refrigerator
Oven capable of reaching 110° C.
Plate Sealer capable of reaching 173° C.
Vortex mixer
Centrifuge (e.g. Eppendorf 5804 R)
BL2+ Biological safety cabinet with integrated germicidal ultraviolet light
Milli-Q Water Purification System

### Procedure

### SARS-CoV-2 Isothermal LAMP by Air Purification Assay (Pre-PCR Room)

### Preparation of Sample Buffer (Biosafety Cabinet)

33.8 mL of PBS was pipetted into a 50 mL conical tube. 230 μL 1% Triton x-100 was added. 450 μL 1M DTT was added. The tube was inverted 10-20×.

### Preparation of LAMP MasterMix (Static Airhoods Clean Room)

The sterile hood, pipettes, and surrounding work areas were wiped down with 10% bleach. Master mixes were set up on a cold block. Each reagent was gently mixed and microfuged prior to pipetting. Two clean 5.0 mL eppendorf tubes were labeled as “Covid” and “RNase P”. Using a clean tip for each reagent, the volume calculated from Table 2 (below) was added for each of the components comprising the master mix for each primer set. The freshly prepared master mix was vortexed and microfuged. Mixes were kept on ice until the Gilson PipetMax robot was ready to add LAMP mixture.

TABLE 1
Overview of Primer Preparation

| | # of Samples | | |
|---|---|---|---|
| (μL) | 16 | 32 | 48 |
| 2.5x SARS-CoV-2 & | 188 | 220.8 | 270.8 |
| 10X RNAseP Primer | | | |
| Stocks | | | |
| Nuclease-Free Water | 712 | 839.2 | 1029.2 |
| Total Volume | 900 | 1060 | 1300 |

TABLE 2
Overview of LAMP Preparation

| | # of Samples | | |
|---|---|---|---|
| (μL) | 16 | 32 | 48 |
| 2X WarmStart LAMP MasterMix | 1000 | 1400 | 1850 |
| LAMP Fluorescent Dye | 40 | 56 | 74 |
| Total Volume | 1040 | 1456 | 1924 |

TABLE 3
Reagent Volumes for Reagent Source Plate

| # of | Sample Buffer | NF Water | Mineral Oil | Beads | Covid | Rnase P | LAMP + Dye |
|---|---|---|---|---|---|---|---|
| Samples | (mL) | (mL) | (mL) | (μL) | (μL) | (μL) | (μL) |
| 16 | 2 | 2 | 1 | 100 | 100 | 100 | 52 |
| 32 | 2 | 2 | 1 | 140 | 120 | 120 | 78 |
| 48 | 2 | 2 | 1 | 200 | 150 | 150 | 104 |

TABLE 4
Reagent Volumes to Add to Source Plate Per Well (For 48 samples)

| | 1 | 2 | 3 | 4 | 5 | 6 | 7 | 8 | 9 | 10 | 11 | 12 |
|---|---|---|---|---|---|---|---|---|---|---|---|---|
| A | Sample | Bead | Sample | Empty | NF- | Mineral | NF- | Mineral | COVID | RNaseP | LAMP + | LAMP + |
| B | Buffer | Mixture | Buffer | | Water | Oil | Water | Oil | Primers | Primers | Dye | Dye |
| C | (2 mL/ | (200 uL/ | (2 mL/ | | (2 mL/ | (1 mL/ | (2 mL/ | (1 mL/ | (150 uL/ | (150 uL/ | Mix | Mix |
| D | well) | well) | well) | | well) | well) | well) | well) | well) | well) | (104 uL/ | (104 uL/ |
| E | | | | | | | | | | | well) | well) |
| F | | | | | | | | | | | | |
| G | | | | | | | | | | | | |
| H | | | | | | | | | | | | |

### Prepare Reagent Plate (Static Air Hoods/Laminar Flow Hood)

Each of the following sequential steps detailing the addition of reagents was conducted in one of the static air hoods/laminar flow hoods. Prior to entry into the hood, the depository was emptied and the ultraviolet light in the static air hood was turned on for 5 minutes. Following UV Sterilization, all of the surfaces and pipettes were wiped down with 10% Bleach.

The Reagent Source Plate was filled with the volumes found in Table 4. Briefly, 2 mL Sample Buffer was added to every well in columns 1 and 3-4 of the Reagent Plate. The bead solution was vortexed for 30 seconds, inverting intermittently, and 200 μL bead solution was added to every well in column 2 of the Reagent Plate. A 6× concentrated bead stock was created by transferring 9.6 mL of well-mixed bead mixture into a 15 mL conical tube. The beads were sedimented by placing a strong magnet (neodymium) on the side of the tube. Once the beads were collected near the magnet, the aqueous portion was slowly removed, being careful not to disturb the beads. The magnet was removed, and beads were resuspended in 1.6 mL of NF water. 2 mL nuclease-free water was added to every well in columns 5 and 7 of the Reagent Plate. 1 mL light mineral oil was added to every well in columns 6 and 8 of the Reagent Plate. A suitable volume from the tube labelled “Covid” was added to each of the wells in column 9 of the Reagent Plate. A volume from the tube labelled “RNase P” was added to each of the wells in column 10 of the Reagent Plate. A volume from the tube labelled “LAMP+Dye” was added to each of the wells in column 11 and 12 of the Reagent Plate.

Reagents were returned to the −20° C. freezer, refrigerator, or room temperature location as appropriate. The now-filled Reagent Source Plate was placed into deck position #3 on the Gilson PipetMax.

Prepare Sample Source Plate (Laminar Flow Biosafety Cabinet)

Each of the following sequential steps detailing the preparation, heat inactivation, and addition of patient samples to the 96 well plate were conducted in one of the laminar flow biosafety cabinets. Prior to entry into hood, the tip depository was emptied and the ultraviolet light in the biosafety cabinet was turned on for 5 minutes.

For each of 48 patient samples, 150 μL of saliva sample was pipetted into different wells in every other column starting with column #1 of the Sample Source Plate (0.5 mL V-Bottom PP Plate, taller plate).

Prepare the Gilson PipetMax

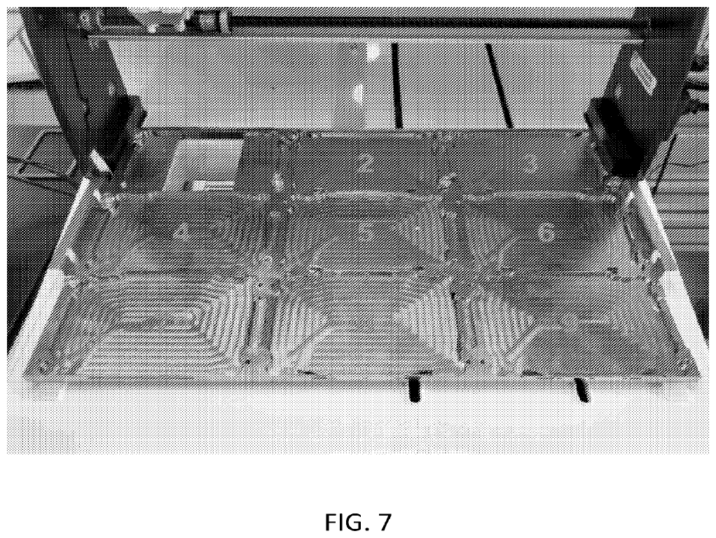

The layout of the Gilson PipetMax is shown in FIG. 7, which may be referred to herein for the specific positions described below.

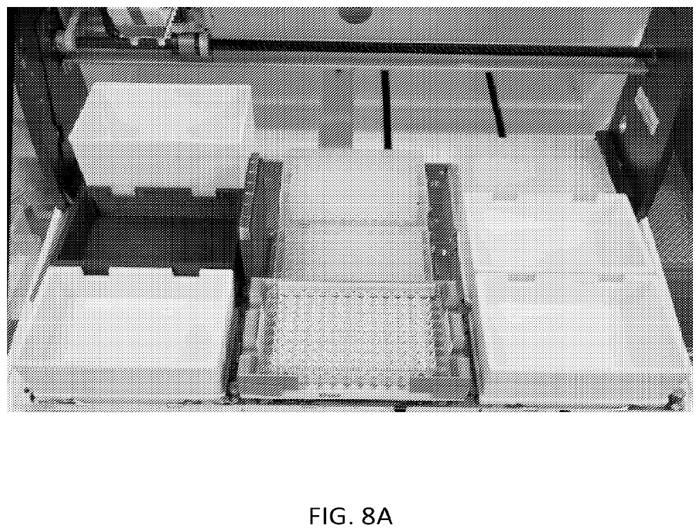

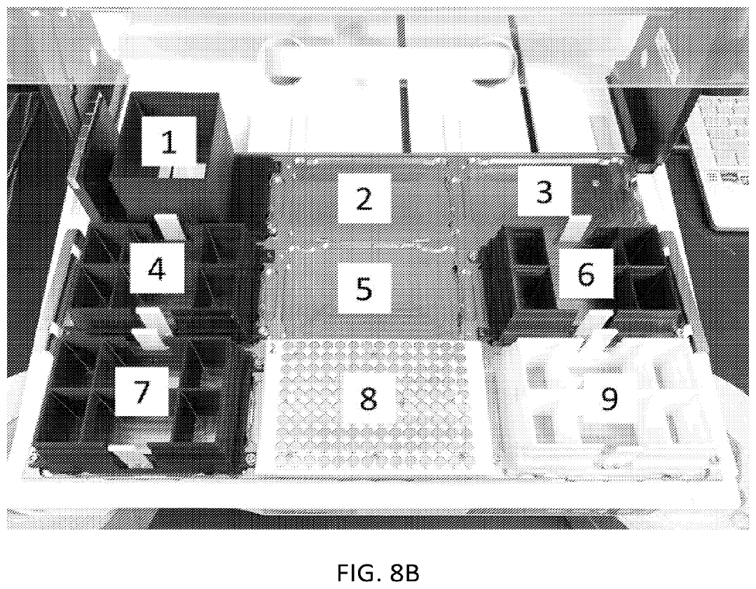

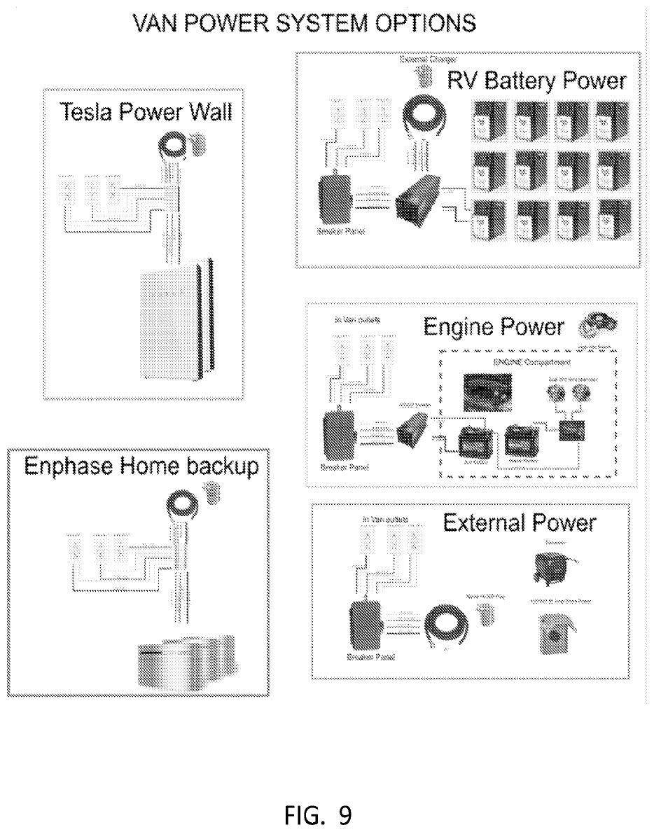

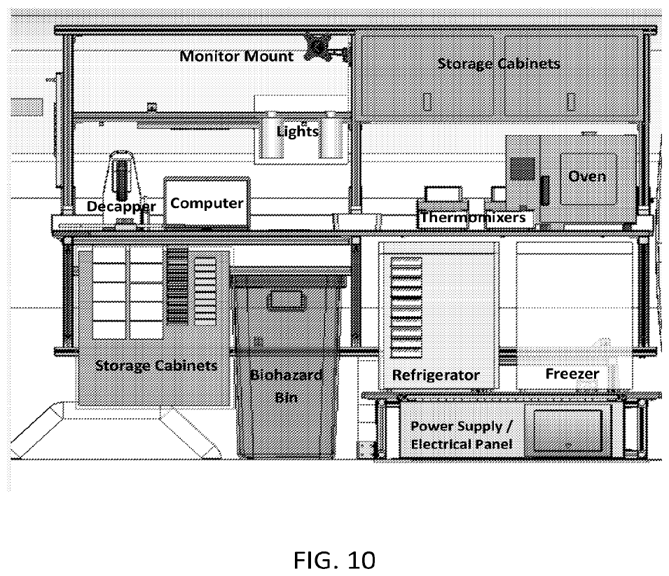

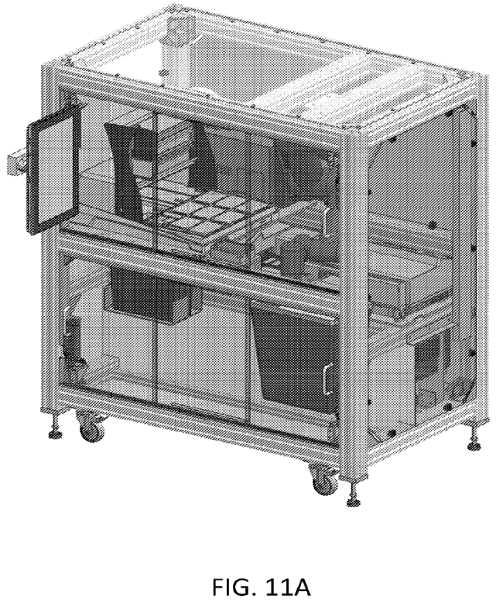

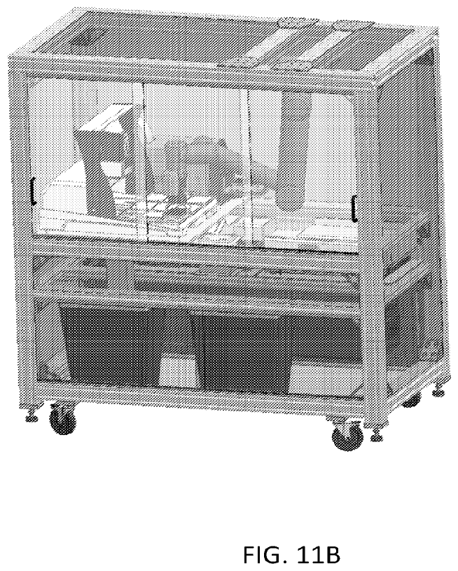

The Tip Disposal Box was placed onto deck position #1. The Reagent Source Plate was secured onto deck position #3. A new Mixing Plate was secured onto deck position #2. The filled Sample Source Plate was secured onto deck position #5, making sure that well A1 was oriented so as to be in the topmost left position of the deck position. A new Wash/Reaction Plate, without the lid, was placed on top of the magnet array (deck position #8) making sure that well A1 was oriented so as to be in the topmost left position of the deck position. The plastic wrap and the container lids were removed from 4 sets of tip boxes. Tip boxes (n=96) were firmly pressed into the tip box cradles (also referred to as “nests”) at positions #4, #6, #7, and #9. The final layout of the prepared Gilson PipetMax is shown in FIG. 8.

The air purification protocol described herein was performed in an automated fashion using a programmed computer. Prior to performing the protocol, it was ensured that all of the on-deck components were placed and oriented correctly, and that all of the non-consumable components (magnetic array, pre-concentration magnet, tip box cradles/nests) were secured to the deck with the included fasteners.

Exemplary Air Purification/LAMP Protocol:

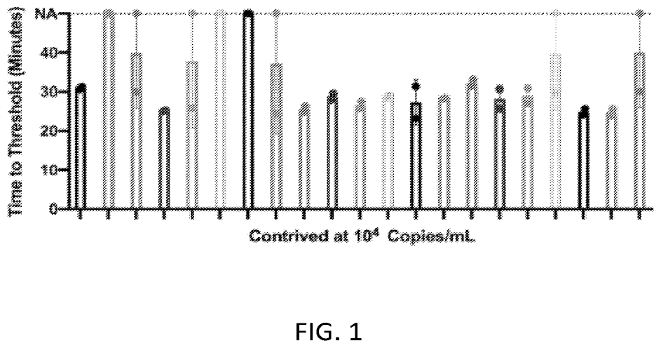

The appropriate volume of the LAMP mixture was added to each well of a multi-well plate (oil/water phases should separate appropriately automatically). The RNA:PMP complexes were purified (separated) from the sample by using a first magnet to generate a concentration of RNA:PMP complexes proximal to the opening of the pipette tips. A second magnet placed under the multi-well plate containing the wash buffer was used to draw the RNA:PMP complexes into the wells. In this case, 96 separate magnets were used, one placed under each of the sample wells. This configuration was discovered to be optimal, although other magnet configurations may be used, including bar magnets (e.g. 8-row bar magnet(s), 16-row bar magnet(s), etc.) and full plate magnets. The wash buffer was aspirated from each well and the target:PMP complexes were allowed to dry. The LAMP mixture was subsequently added to each well. LAMP was performed and results were analyzed using software to run desired algorithms to determine endpoints (e.g. positive, negative). Fluorescence was quantified using arbitrary fluorescent “read” intervals that generate a standard sigmoidal amplification curve. From this curve, proprietary R analysis calculates time-to-threshold values. While the time-to-threshold values are useful for troubleshooting purposes, the results of LAMP are assessed as binary, wherein detection of a signal above threshold indicates a positive result (e.g. presence of the pathogen) and no detection or detection below threshold indicates a negative result. Time to threshold values for contrived samples are shown in FIG. 1. The automatic and robotic systems may also be configured for semi-quantitative and quantitative assays, in addition to qualitative, “yes-no” assays, if desired.

### Example 2

### Timing of DTT Addition

Efficacy sample extraction in terms of time to threshold for the LAMP assay was evaluated in samples with DTT added before heating the saliva and DTT added after heating the saliva, respectively. Samples were treated as generally described in Example 1. Saliva samples were diluted in Triton X-100 (1%), DTT (1M), paramagnetic beads (Cytiva beads, 20 μL/well), and PBS for a total volume of 420 μL. The wash volume was 160 μL. For the LAMP reaction, 12.5 μL master mix, 2.5 μL primers (to the N-gene and As1e of SARs-CoV2), and 0.5 μL dye were used along with 9.5 μL H₂O for a total volume in each well of 25 μL.

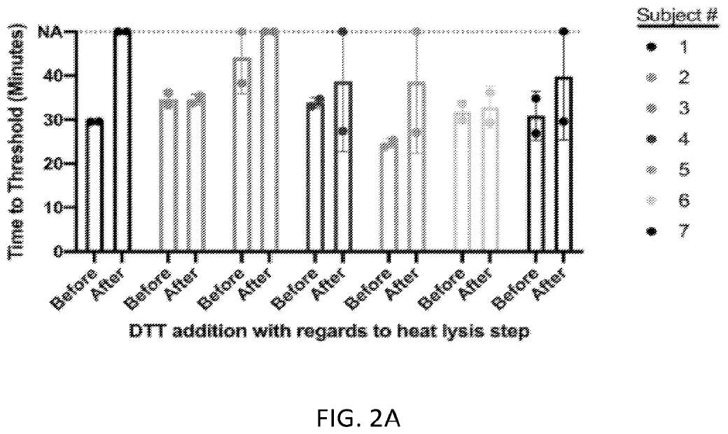

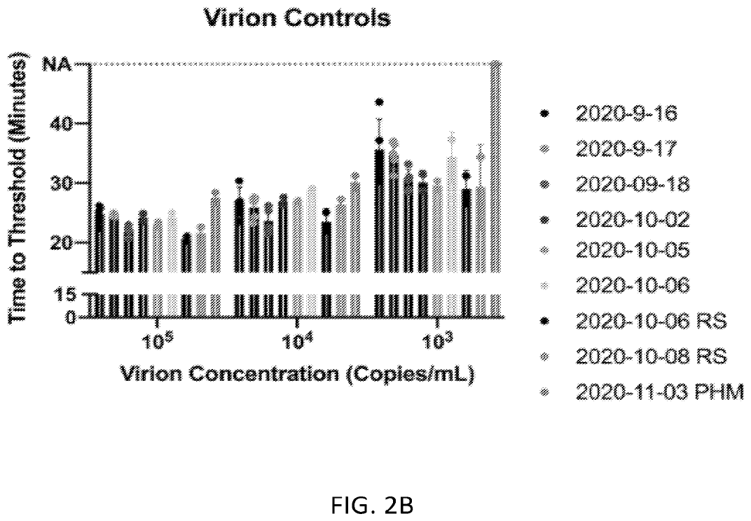

Results comparing timing of DTT addition are shown in FIG. 2A. Results show that addition of DTT before the heat lysis step decreases the time to threshold values for each sample. FIG. 2B shows comparative results from virion controls.

### Example 3

### Effect of Triton Addition

The present example highlights the viscosity, e.g. “stickiness” of saliva and demonstrates the needs for appropriate sample handling to enable accurate quantification of the target in samples.

Experimenters noticed beads sticking to the tube and tips when processing patient samples. Various reagents such as surfactants were added during processing to try to minimize sticking and thereby improve accuracy of detection. These changes were done while also considering the impact of the reagents on surface tension of the liquid/air boundary and transfer of PMI's via magnetic force. In this case, addition of too much surfactant in the patient sample itself inhibits binding while insufficient surfactant results in clumping.

Samples: Contrived saliva and NP swabs in UTM, PBS (pH 7.4), and saline
Template: gamma-irradiated SARS CoV-2

### Conditions:

40 ul of saliva or 80 uL NP media transferred directly to sample buffer containing:
   0.005-0.01% triton
   10 mM DTT (except for UTM conditions)
   10 uL Cytiva paramagnetic beads
   210 uL total volume (PBS)
   180 uL wash volume
   Round Bottom Plate
25 uL LAMP reaction
   12.5 ul MM—(Warmstart 2× MasterMix)
   2.5 uL each primer set
   0.5 uL Dye
   25 uL total volume
Plate Reader
   60 second cycles
   51 minutes total

Saliva or NP samples were mixed with sample buffer to generate target-PMP complexes. A hybrid interface was generated by dipping the pipette tips into light mineral oil, pulling the tips into the air, and aspirating about 84, of air into the tip. Complexes were concentrated near the interface using a first magnet. Following concentration, target-PMP complexes were drawn into wells of a wash plate containing 18 μL wash buffer. Wash buffer was aspirated from the wells and the target-PMP complexes contained therein were allowed to dry.

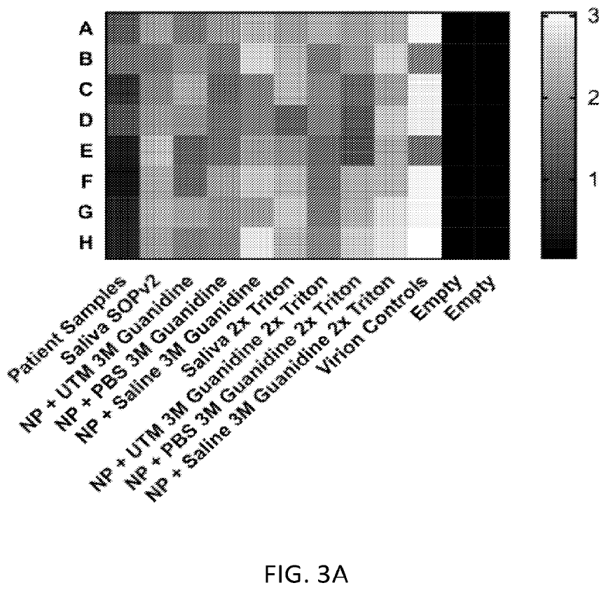

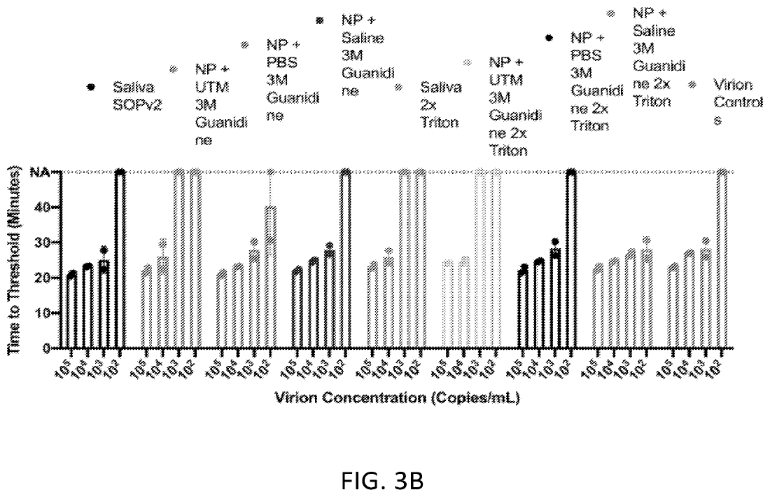

RT-LAMP reagents were added to the wells. For the LAMP reaction, 12.5 μL master mix, 2.5 μL of each 10× primer stock (to the N-gene and As1e of SARs-CoV2), and 0.5 μL dye were used along with 7 μL H₂O for a total volume in each well of 25 μL. RT-LAMP was performed using a plate reader and results were quantified. Results are shown in FIG. 3A-3B. FIG. 3A shows a heatmap of saliva or NP samples proceed with different sample buffer reagents. FIG. 3B shows the results for the same combinations, quantified as time to threshold values.

### Example 4

### Exemplary NP Swab Protocol

Conditions
   40 uL or 60 uL NP sample in UTM, PBS, or saline
   NP sample transferred directly to sample buffer containing:
      0.5% triton
      10 uL of each Cytiva bead
      210 uL total volume (PBS)
   180 uL wash volume
   120 uL Oil Overlay
   Round Bottom Plate
   25 uL LAMP reaction
   12.5 ul MM
   2.5 uL each primer set
   0.5 uL Dye
   25 uL total volume

NP samples were mixed with sample buffer to generate target-PMP complexes. A hybrid interface was generated by dipping the pipette tips into light mineral oil, pulling the tips into the air, and aspirating about 84, of air into the tip. Complexes were concentrated near the interface using a first magnet. Following concentration, target-PMP complexes were drawn into wells of a wash plate containing 180 μL wash buffer. Wash buffer was aspirated from the wells and the target-PMP complexes contained therein were allowed to dry.

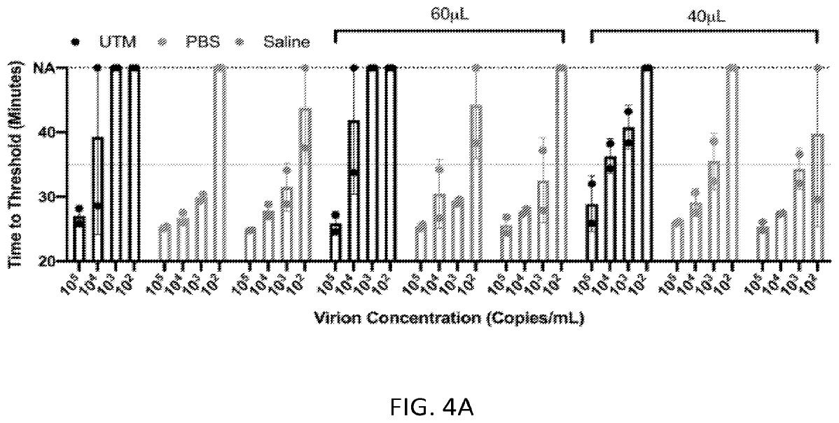

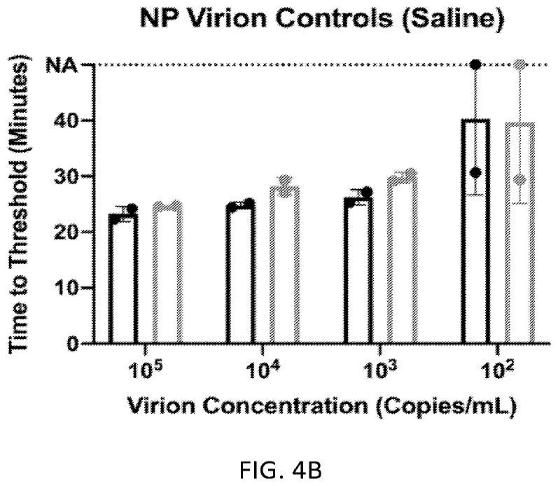

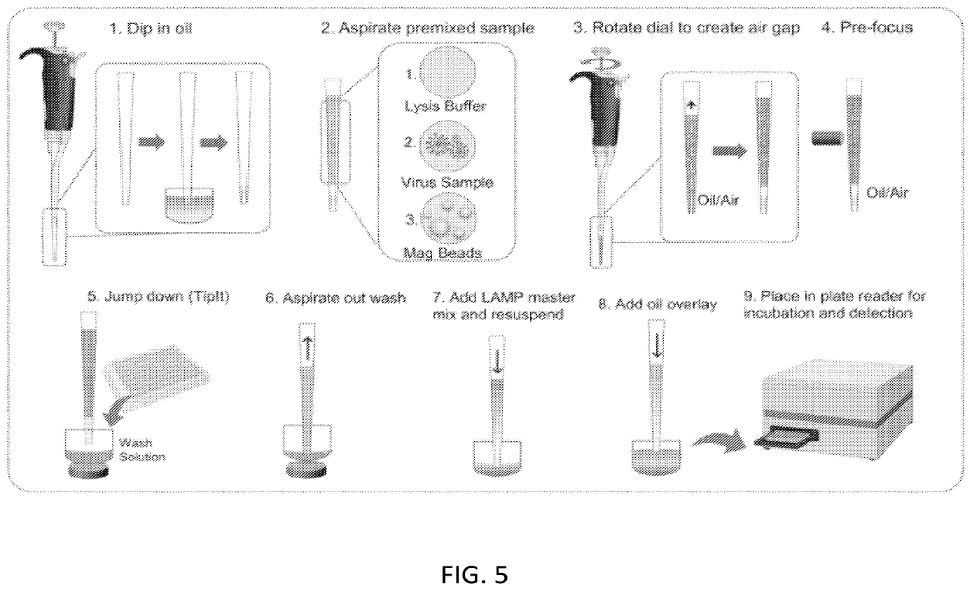

RT-LAMP reagents were added to the wells. For the LAMP reaction, 12.5 μL master mix, 2.5 μL of each primer (to the N-gene and As1e of SARs-CoV2), and 0.5 μL dye were used along with 7 μL H₂O for a total volume in each well of 25 μL. RT-LAMP was performed using a plate reader and results were quantified. Results are shown in FIG. 4A-4B. FIG. 4A shows time to threshold values for NP samples in various buffers (UTM, PBS, or saline). Time to threshold values for NP virion controls in saline are shown in FIG. 4B.

1. A mobile laboratory comprising:
a) a computer system comprising one or more processors, said processors configured to: i) catalog a plurality of biological samples collected from distinct subjects; ii) operate a robotic sample processing system; iii) operate a robotic sample analysis system; iv) associate data received from said sample analysis system with individual members of said plurality of biological samples; and v) communicate said data;
b) a robotic sample processing system configured to (i) mix each biological sample with paramagnetic particles (PMPs) to generate a composition comprising one or more target-PMP complexes, and (ii) isolate target-PMP complexes from each composition; and
c) a robotic sample analysis system configured to receive target-PMP complexes and carry out an assay to detect said target, if present.

2. The mobile laboratory of claim 1, wherein said biological samples comprise nasopharyngeal samples, oropharyngeal samples, oral swab samples, oral sponge samples, nasal swab samples, mid-turbinate samples, or saliva samples, and wherein said biological samples are stored in a sample storage container comprising a storage bugger.

3. (canceled)

4. (canceled)

5. The mobile laboratory of claim 1, wherein the sample storage container is heated to at least 40° C. after placement of the biological sample within the container.

6. The mobile laboratory of claim 1, wherein the biological sample additionally comprises a reducing agent and/or a protease.

7. (canceled)

8. (canceled)

9. (canceled)

10. (canceled)

11. (canceled)

12. The mobile laboratory of claim 1, wherein the PMPs are contained in a liquid composition, wherein the liquid composition optionally comprises a reducing agent and/or a detergent.

13. (canceled)

14. (canceled)

15. The mobile laboratory of claim 1, wherein the PMPs are contained in a lyophilized formulation.

16. The mobile laboratory of claim 1, wherein the robotic sample processing system comprises a multichannel pipette and an apparatus for operating the multichannel pipette, wherein the apparatus for operating the multichannel pipette is configured to induce movement of the multichannel pipette and to aspirate and/or inject liquid when pipette tips are attached to the multichannel pipette.

17. (canceled)

18. (canceled)

19. The mobile laboratory of claim 16, wherein the apparatus for operating the multichannel pipette is controlled by said computer system.

20. The mobile laboratory of claim 16, wherein mixing each biological sample with PMPs is performed in a multi-well plate using a plurality of pipette tips attached to the multichannel pipette.

21. The mobile laboratory of claim 16, wherein isolating the target-PMP complexes from each composition comprises:
a. Aspirating each composition into a distinct pipette tip; and
b. Positioning the multichannel pipette proximal to a magnet positioned below a sample collection device, such that the magnet draws the target-PMP complexes out of the pipette tips and into the sample collection device.

22. The mobile laboratory of claim 16, wherein isolating the target-PMP complexes from each composition comprises:
a. Aspirating each biological sample into a distinct pipette tip containing lyophilized PMPs to generate a composition comprising one or more target-PMP complexes within each pipette tip; and
b. Positioning the multichannel pipette proximal to a magnet positioned below a sample collection device, such that the magnet draws the target-PMP complexes out of the pipette tips and into the sample collection device.

23. The mobile laboratory of claim 16, wherein isolating the target-PMP complexes from each composition comprises:
a. Aspirating each composition into a distinct pipette tip;
b. Generating a liquid/air interface, a liquid/oil interface, or a hybrid interface proximal to a bottom opening of the pipette tip; and
c. Positioning the multichannel pipette proximal to a magnet positioned below a sample collection device, such that the magnet draws the target-PMP complexes through the liquid/air interface, liquid/oil interface, or hybrid interface and into the sample collection device.

24. The mobile laboratory of claim 16, wherein isolating the target-PMP complexes from each composition comprises:
a. Aspirating each composition into a distinct pipette tip;
b. Generating a liquid/air interface, a liquid/oil interface, or a hybrid interface proximal to a bottom opening of the pipette tip;
c. Positioning the multichannel pipette proximal to a first magnet to generate a concentration of target-PMP complexes proximal to the liquid/air interface, liquid/oil interface, or hybrid interface; and
d. Positioning the multichannel pipette proximal to a second magnet to draw the target-PMP complexes through the liquid/air interface, liquid/oil interface, or hybrid interface and into a sample collection device.

25. The mobile laboratory of claim 16, wherein isolating the target-PMP complexes from each composition comprises:
a. Aspirating each biological sample into a distinct pipette tip containing lyophilized PMPs to generate a composition comprising one or more-target PMP complexes within each pipette tip;
b. Generating a liquid/air interface, a liquid/oil interface, or a hybrid interface proximal to a bottom opening of the pipette tip; and
c. Positioning the multichannel pipette proximal to a magnet positioned below a sample collection device, such that the magnet draws the target-PMP complexes through the liquid/air interface, liquid/oil interface, or hybrid interface and into the sample collection device.

26. The mobile laboratory of claim 16, wherein isolating the target-PMP complexes from each composition comprises:
a. Aspirating each biological sample into a distinct pipette tip containing lyophilized PMPs to generate a composition comprising one or more-target PMP complexes within each pipette tip;
b. Generating a liquid/air interface, a liquid/oil interface, or a hybrid interface proximal to a bottom opening of the pipette tip;
c. Positioning the multichannel pipette proximal to a first magnet to generate a concentration of target-PMP complexes proximal to the liquid/air interface, liquid/oil interface, or hybrid interface; and
d. Positioning the multichannel pipette proximal to a second magnet to draw the target-PMP complexes through the liquid/air interface, liquid/oil interface, or hybrid interface and into a sample collection device.

27. The mobile laboratory of claim 26, wherein (a) each composition or biological sample is aspirated into a distinct pipette tip through the bottom opening of the pipette tip, and wherein generating the liquid/air interface, liquid/oil interface, or hybrid interface comprises further aspirating the composition within the pipette tip while the bottom opening of the pipette tip is exposed to air, or (b) each composition or biological sample is aspirated into a distinct pipette tip through a side opening of the pipette tip while the bottom opening of the pipette tip is in conformal contact with a surface such that liquid is unable to enter the pipette tip through the bottom opening, thereby generating the liquid/air interface, liquid/oil interface, or hybrid interface.

28. (canceled)

29. The mobile laboratory of claim 26, wherein the sample collection device comprises a multi-well plate, such that the target-PMP complexes from each composition are drawn into a distinct well on the multi-well plate.

30. The mobile laboratory of claim 27, wherein the system further comprises a moveable surface, wherein the biological samples, one or more magnets, and/or the sample collection device are housed on the moveable surface, and wherein the moveable surface is configured to change orientation and/or move in the x-y plane and/or in the vertical z-direction.

31. (canceled)

32. The mobile laboratory of claim 26, wherein the distinct wells on the multi-well plate comprise a wash buffer.

33. The mobile laboratory of claim 32, wherein processing the plurality of biological samples further comprises aspirating the wash buffer from the wells and allowing the target-PMP complexes contained therein to dry.

34. The mobile laboratory of claim 27, wherein the assay comprises reagents for loop-mediated isothermal amplification (LAMP)-based detection or quantitation of the target; (b) the target comprises a viral nucleic acid, including one or more viral nucleic acid targets selected from the group consisting of coronavirus, rhinovirus, influenza, respiratory syncytial virus, adenovirus, parainfluenza, human immunodeficiency virus, human papillomavirus, rotavirus, hepatitis C virus, zika virus, Ebola virus, tuberculosis, Borrelia burgdorferi, staphylococcus, aspergillus, Streptococcus pyrogenes and SARS-CoV2 nucleic acid; and (c) target-PMP complexes are contacted with the reagents for LAMP-based detection or quantification of the target, and a signal resulting from contact is displayed and/or measured, wherein the signal may be a colorimetric signal or a fluorescent signal and data regarding detection of the target comprises data regarding the signal.

35. (canceled)

36. (canceled)

37. (canceled)

38. (canceled)

39. (canceled)

40. (canceled)

41. The mobile laboratory of claim 21, wherein the data regarding detection of the target is correlated with a unique identifier for each biological sample.

42. The mobile laboratory of claim 23, wherein the mobile laboratory is housed within a vehicle.

43. The mobile laboratory of claim 27, wherein the mobile laboratory is housed within a building for a period of less than 6 months prior to being moved to a new location.
